SEQUENCE LISTING (5 sequences)

SEQ ID NO: 1 - RNA, 20 nt, Homo sapiens
aguaucuuugcacaaaccag

SEQ ID NO: 2 - RNA, 20 nt, Mus musculus
aguaucuuugcacaaaccag

SEQ ID NO: 3 - RNA, 20 nt, Rattus norvegicus
aguaucuuugcacaaaccag

SEQ ID NO: 4 - RNA, 20 nt, Canis familiaris
aguaucuuugcacaaaccag

SEQ ID NO: 5 - RNA, 20 nt, Unknown
  Description of Unknown Conserved consensus binding site for miR-19s of the 3′UTR of SOCS-1
aguaucuuugcacaaaccag

(19) United States
(12) Patent Application Publication
(10) Pub. No.: US 2011/0152357 A1
(43) Pub. Date: Jun. 23, 2011

(54) Micro-RNA-Based Compositions and Methods for the Diagnosis, Prognosis and Treatment of Multiple Myeloma

(75) Inventor: Carlo M. Croce, Columbus, OH (US)

(73) Assignee: THE OHIO STATE UNIVERSITY, Columbus, OH (US)

(21) Appl. No.: 13/058,662

(22) PCT Filed: Aug. 12, 2009
(86) PCT No.: PCT/US2009/053586

§ 371 Date: Feb. 24, 2011

Related U.S. Application Data
(60) Provisional application No. 61/088,036, filed on Aug. 12, 2008.

(51) Int. Cl. A61K 31/7088; C12Q 1/68; C40B 30/04; C07H 21/02; C07K 16/00; G01N 33/574; C12N 5/071; A61P 35/00

(52) U.S. Cl. 514/44 A; 435/6.14; 506/9; 514/44 R; 536/23.1; 530/389.1; 435/7.1; 435/375

(57) ABSTRACT

Methods for assessing a pathological condition in a subject includes measuring an expression profile of one or more markers where a difference is indicative of multiple myeloma (MM) or a predisposition to MM. The present invention provides novel methods and compositions for the diagnosis, prognosis and treatment of MM. The invention also provides methods of identifying anti-MM cancer agents.

### CROSS-REFERENCE TO RELATED APPLICATIONS

This application claims the benefit of U.S. Provisional Application No. 61/088,036 filed Aug. 12, 2008, the entire disclosure of which is expressly incorporated herein by reference.

### STATEMENT REGARDING FEDERALLY SPONSORED RESEARCH

This invention was made with government support under National Cancer Institute Grant No. ______. The government has certain rights in this invention.

### TECHNICAL FIELD AND INDUSTRIAL APPLICABILITY OF THE INVENTION

This invention relates generally to the filed of molecular biology. More particularly, it concerns methods and compositions involving biomarkers for multiple myeloma. Certain aspects of the invention include application in diagnostics, therapeutics, and prognostics of multiple myeloma related diseases.

### BACKGROUND OF THE INVENTION

There is no admission that the background art disclosed in this section legally constitutes prior art.

Multiple myeloma (MM) is a B-cell neoplasm characterized by the accumulation of clonal malignant plasma cells in the bone marrow (1). This cancer can occur de novo or develop from a benign condition called monoclonal gammopathy of undetermined significance (MGUS) at a rate of approximately 1% per year (2-3). MM cells are endowed with a multiplicity of antiapoptotic signaling mechanisms, which account for resistance to current chemotherapy regimens (4). Therapeutic modalities that are effective in MM modulate levels of the proapoptotic and antiapoptotic Bcl-2 family of proteins and of inhibitors of apoptosis, which are primarily regulated by p53 (mutated at low frequency in MM) (4). It is well known that the bone marrow (BM) microenvironment plays a prominent role in the biology of MM; adhesion of MM cells to the BM stroma triggers cytokines production, enhances cell proliferation and resistance to chemotherapy by activation of nuclear factor lines (7), and more importantly it seems that is the major genetic difference between MM and MGUS (3-7).

Despite recent advances in oncogenomics and MM cell-stroma interactions, there is an urgent need to identify critical players in MM pathogenesis that could be targeted by pharmacological intervention to improve outcome for this still incurable disease. The advent of new techniques, such as microarray gene expression, including non coding RNAs, may lead to an improved understanding of MM biology by establishing associations between gene expression changes and MM molecular and clinical features as shown by us for chronic lymphoid and acute myelogenous leukemia (8-9).

MicroRNAs (miRNAs) are non-coding RNAs of 19-25 nucleotides in length that regulate gene expression by inducing translational inhibition and degradation of their target mRNAs through base pairing to partially or fully complementary sites (10). MiRNAs are involved in critical biological processes, including development, cell differentiation, stress response, apoptosis and proliferation (10). Recently, specific miRNA expression patterns have been linked to hematopoiesis and cancer (11-13).

Little is known, however, about miRNA expression in MM. A recent study has shown that, in IL-6 dependent MM cell lines, miR-21 transcription is controlled by IL-6 through a STAT-3 mechanism. Moreover, ectopic miR-21 expression is sufficient to sustain growth of IL-6-dependent cell lines in the absence of IL-6 (14).

In view of such, there is a need for a method for reliably and accurately diagnosing and/or screening individuals for a predisposition to multiple myeloma (MM)

A method of treating multiple myeloma (MM) associated cancers associated therewith is also highly desired.

In spite of considerable research into therapies for multiple myeloma (MM), MM remains difficult to diagnose and treat effectively, and the mortality observed in patients indicates that improvements are needed in the diagnosis, treatment and prevention of the disease.

### SUMMARY OF THE INVENTION

In a first broad aspect, there is provided herein a method for assessing a pathological condition in a subject includes measuring an expression profile of one or more markers where a difference is indicative of multiple myeloma (MM) or a predisposition to MM.

In another broad aspect, there is provided herein novel methods and compositions for the diagnosis, prognosis and treatment of MM. The invention also provides methods of identifying anti-MM cancer agents.

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file contains one or more drawings executed in color and/or one or more photographs. Copies of this patent or patent application publication with color drawing(s) and/or photograph(s) will be provided by the Patent Office upon request and payment of the necessary fee.

FIGS. 1A-1D. MM and MGUS express a distinct spectrum of miRNA in comparison to normal CD138+ PCs:

FIG. 1A: Schematic drawing showing the multistep molecular process of PCs transformation.

FIG. 1B: Representative list of the miRNAs significantly deregulated in MGUS versus normal PCs analyzed. The asterisks indicate the specific associated cluster.

FIG. 1C: Representative list of the common deregulated miRNAs in the comparison classes MM patient vs. normal PCs and PCs MM vs. normal PCs analyzed. The asterisks indicate the specific associated cluster.

FIG. 1D: miR-17 microRNA clusters. Three paralog families of microRNA precursors can be identified: miR-17/18/18X120/93/106a/106b/93 (yellow), miR-19a/19b-1/19b-2 (blue) and miR-92-1/92-2/25 (green).

FIGS. 2A-2D: miR-181s, 106b-25 cluster, 32 target PCAF:

FIG. 2A: miRNAs predicted to interact with PCAF gene in several consensus binding sites at its 3′-UTR, according to “in silico” target Target Scan prediction software.

FIGS. 2B, 2C, 2D: Luciferase assay showing decreased luciferase activity in cells cotransfected with pGL3-PCAF-3* activity±SD. Each reporter plasmid was transfected at least twice (on different days) and each sample was assayed in triplicate.

FIG. 2D: Western blot showing SOCS-1 protein in whole cell lysates from U266 and JJN3 cells at 48 hrs after transfection with scrambled oligonucleotides or miR-19a, miR-19b, or both ASOs. GADPH was used as loading control. Densitometry based on GADPH levels shows increased level of SOCS-1 in presence of miR-19a or miR-19b or together anti-sense oligos (ASOs) in U266 and JJN3 cells.

FIG. 2E: Western blot showing that miR-19s modulate expression of activated STAT-3 in U266 cells in vitro. Cells were transfected with anti-miR-19s or negative control (Scr) miRNAs inhibitors in vitro, and cell lysates were obtained after 72 h. Densitometry based on STAT-3 levels shows decreased level of P-STAT-3 in presence of miR-19s.

FIG. 2F: Stem-loop qRT-PCR to validate the expression of endogenous miR-19a and miR-19b, at 72 hrs after transfection of U266 cells (FIGS. 2G-3H).

FIG. 2I: Real-time RT-PCR analyses for p53 and PCAF expression in MM.1s cells transfected with miR-181a/b or with miR-181a/b and miR-106b/25 together ASOs (pool) or with scrambled oligonucleotide at 48 hrs after transfection and after 4 hr of UV treatment. The PCR products for both genes were normalized to GADPH and ACTIN expression. The bar-graph represents the mean values observed in four separate studies±SE.

FIG. 2L: Immununoblot analysis showing p53 protein expression after 48 hrs of miR-181a, miR-181b, miR-92 and scrambled ASOs transfection in MM1 cells after 9 hrs and overnight incubation with 10 μM nutlin-3a; GADPH was internal loading control. Densitometry based on GADPH levels shows increased level of p53 in presence of miR-181a and miR-181b ASOs in MM.1s cells.

FIGS. 3A-3F: miR-19s target SOCS-1 in MM cell lines:

FIG. 3A: Immunoblot analysis with antisera against SOCS-1 and GADPH in 15 MM cell lines and two CD138+PCs from healthy donors (control).

FIG. 3B: Predicted highly conserved consensus binding site in human, mouse, rat and dog for miR-19s on the 3′UTR of SOCS-1.

FIG. 3C: Relative luciferase activity in MEG01 cells transiently cotransfected with luciferase reporter vector containing the 3′ UTR of SOCS-1 and miR-19s or scrambled oligonucleotides. Deletion of the six bases in the putative miR-19s binding site, complementary to miRNA seed region, abrogates this effect (MUT). Bars indicate firefly luciferase activity normalized to renilla luciferase activity±SD. Each reporter plasmid was transfected at least twice (on different days) and each sample was assayed in triplicate.

FIG. 3D: Western blot showing SOCS-1 protein in whole cell lysates from U266 and JJN3 cells at 48 hrs after transfection with scrambled oligonucleotides or miR-19a, miR-19b, or both ASOs. GADPH was used as loading control. Densitometry based on GADPH levels shows increased level of SOCS-1 in presence of miR-19a or miR-19b or together ASOs in U266 and JJN3 cells.

FIG. 3E: Western blot showing that miR-19s modulate expression of activated STAT-3 in U266 cells in vitro. Cells were transfected with anti-miR-19s or negative control (Scr) miRNAs inhibitors in vitro, and cell lysates were obtained after 72 h. Densitometry based on STAT-3 levels shows decreased level of P-STAT-3 in presence of miR-19s.

FIG. 3F: Stem-loop qRT-PCR to validate the expression of endogenous miR-19a and miR-19b, at 72 hrs after transfection of U266 cells with antagonizing oligonucleotides, after normalization with RNU6B.

FIGS. 4A-4B: Antagonizing miR-19s and miR-181s expression in MM cell lines resulted in significant tumor suppression in nude mice. U266 and JJN3 cells (30×106 cells) were injected subcutaneously miR-181s ASOs or scrambled oligo. Mice were sacrificed on day 35 and tumor volumes were calculated.

FIG. 4A: Time course of tumor growth of U266 cell line.

FIG. 4B: Time course of tumor growth of JJN3 cell line; tumors treated with miR-19s and miR-181s ASOs were significantly smaller than tumors of scrambled groups for both cell lines (scale bar; 10 mm).

FIGS. 5A-5C: Common miRNAs expression. A Venn Diagram showing the common miRNAs (FIG. 5C) between the two class of comparison MM PCs versus healthy PCs (FIG. 5A) and PCs from MM patients versus healthy PCs (FIG. 5B).

FIGS. 6A-6F. Validation of microarray data in MM patients, MGUS and MM cell lines vs CD138+PCs healthy by qRT-PCR. Average miR-93, miR-25 and miR-106b (FIG. 6A), miR-181a and miR-181b (FIG. 6B), miR-32 (FIG. 6C), miR-17-5 and miR-20a (FIG. 6D) miR-92 and miR-106a (FIG. 6E) and miR-19a and miR-19b (FIG. 6F) expression in CD138+PCs from healthy donors (n=3), from MGUS (n=3), from MM patients (n=6) and MM cell lines (n=15) measured by qRT-PCR. Bars represent relative fold-changes between MM PCs, MGUS and healthy PCs±SE. The miRNA expression between the different groups was compared after normalization with RNU6B.

FIGS. 7A-7B: PCAF expression in MM cell lines:

FIG. 7A: Real-time RT-PCR analyses for PCAF expression in 15 MM cell lines and 1 healthy CD138+PC sample. The PCR product was normalized to GADPH and ACTIN expression and each point was repeated in quadruplicate; differences were significant (p<0.001).

FIG. 7B: Immunoblot analysis of 15 MM cell lines showing expression of PCAF normalized for ACTIN expression.

FIGS. 8A-8B: Validation of ASOs transfection:

FIG. 8A: Immunoblot analysis showing PCAF protein expression in MM.1s cells at 48 hrs after miR-181a/b, miR-106b-25 cluster, miR-32 (pool) or miR-181a/b or scrambled ASOs transfection in MM1 cells; GADPH was internal loading control.

FIG. 8B: Stem-loop q-RT-PCR to validate the expression of endogenous miR-181a/b, miR-106b-25 cluster, miR-32 at 48 hrs after transfection of MM.1s cells with antagonizing oligonucleotides, after normalization with U6.

FIG. 9: miR-17-92 cluster targets Bim. Immunoblot analyses showing BIM-EL, BIM-L and GADPH expression at 48 hrs after transfection with miR-19a or miR-19b or together ASOs in U266 cells. Densitometry based on GADPH levels shows increased level of BIM-EL and BIM-L in presence of miR-19b and miR-19a and b together ASOs in U266 cells.

FIG. 10: Table 1—Patient samples clinical data.

FIG. 11: Table 2—miRNAs differentially expressed between MGUS versus healthy PCs.

FIG. 12: Table 3—MM cell lines use for microarray analysis and stem-loop.

FIG. 13: Table 4—miRNAs differentially expressed between MM patients and cell lines (MM PCs) versus healthy PCs.

FIG. 14: Table 5—miRNAs differentially expressed between PCs from MM patients versus healthy PCs.

FIG. 15: Table 6—Common miRNAs expressed between MM PCs and MM patients.

### DETAILED DESCRIPTION OF THE PREFERRED EMBODIMENT

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

In a broad aspect, there is provided herein a method of diagnosing whether a subject has, or is at risk for developing, multiple myeloma (MM) and/or monoclonal gammopathy of undetermined significance (MGUS), comprising: measuring the level of at least one miR gene product in a test sample from the subject, wherein an alteration in the level of the miR gene product in the test sample, relative to the level of a corresponding miR gene product in a control sample, is indicative of the subject either having, or being at risk for developing, MM and/or MGUS.

In certain embodiments, the at least one miR gene product is one or more of: miR-21, miR-25, miR-106b˜25 cluster, miR-181a, miR-181b, miR-106a, miR-17˜92 cluster, miR-19a, miR-19b and miR-32. In certain embodiments, the at least one miR gene product is one or more of: miR-21, miR-19a, miR-19b, miR-181a, miR-181b and miR-32. In certain embodiments, the at least one miR gene product is one or more of miR-19a and miR-19b.

In certain embodiments, the at least one miR gene product is indicative of the subject having MM, as distinguished from MGUS.

In another broad aspect, there is provided herein, a method for suppressing tumor growth in a subject in need thereof, comprising administering at least one gene product is one or more of: miR-21, miR-25, miR-106b˜25 cluster, miR-181a, miR-181b, miR-106a, miR-17˜92 cluster, miR-19a, miR-19b and miR-32. In certain embodiments, the at least one miR gene product is one or more of: miR-19a, miR-19b, miR-181a and miR-181b. the miR gene product is one or more of: miR-191a and miR-191b.

In certain embodiments, at least one miR gene product is associated with p53 protein regulation.

In another broad aspect, there is provided herein, a method of diagnosing whether a subject has, or is at risk for developing, multiple myeloma (MM), comprising: 1) reverse transcribing RNA from a test sample obtained from the subject to provide a set of target oligodeoxynucleotides; 2) hybridizing the target oligodeoxynucleotides to a microarray comprising miRNA-specific probe oligonucleotides to provide a hybridization profile for the test sample; and (3) comparing the test sample hybridization profile to a hybridization profile generated from a control sample, wherein an alteration in the signal of at least one miRNA is indicative of the subject either having, or being at risk for developing, the MM disease.

In another broad aspect, there is provided herein, a method of diagnosing whether a subject has, or is at risk for developing, a multiple myeloma (MM) related disease associated with one or more adverse prognostic markers in a subject, comprising: 1) reverse transcribing RNA from a test sample obtained from the subject to provide a set of target oligodeoxynucleotides; 2) hybridizing the target oligodeoxynucleotides to a microarray comprising miRNA-specific probe oligonucleotides to provide a hybridization profile for the test sample; and 3) comparing the test sample hybridization profile to a hybridization profile generated from a control sample, wherein an alteration in the signal is indicative of the subject either having, or being at risk for developing, the MM related disease.

In another broad aspect, there is provided herein, a method of treating a multiple myeloma (MM) related disease in a subject suffering therefrom in which at least one miR gene product is down-regulated or up-regulated in MM cells of the subject relative to control cells, comprising: 1) when the at least one miR gene product is down-regulated in the MM cells, administering to the subject an effective amount of at least one isolated miR gene product, such that proliferation of MM cells in the subject is inhibited; or 2) when the at least one miR gene product is up-regulated in the MM cells, administering to the subject an effective amount of at least one compound for inhibiting expression of the at least one miR gene product, such that proliferation of MM cells in the subject is inhibited.

In another broad aspect, there is provided herein, a method of treating multiple myeloma (MM) related disease in a subject, comprising: 1) determining the amount of at least one miR gene product in MM cells, relative to control cells; and 2) altering the amount of miR gene product expressed in the MM cells by: i) administering to the subject an effective amount of at least one isolated miR gene product, if the amount of the miR gene product expressed in the MM cells is less than the amount of the miR gene product expressed in control cells; or ii) aministering to the subject an effective amount of at least one compound for inhibiting expression of the at least one miR gene product, if the amount of the miR gene product expressed in the MM cells is greater than the amount of the miR gene product expressed in control cells, such that proliferation of MM cells in the subject is inhibited. In certain embodiments, the at least one miR gene product is indicative of the subject having MM, as distinguished from MGUS.

In another broad aspect, there is provided herein, a pharmaceutical composition for treating a multiple myeloma (MM) related disease, comprising at least one isolated miR gene product and a pharmaceutically-acceptable carrier. In certain embodiments, the miR gene product comprises at least one or more of: miR expression inhibitors and anti-sense oligos (ASOs). In certain embodiments, the at least one miR expression inhibitor compound is specific for a miR gene product that is up-regulated in MM cells relative to suitable control cells.

In another broad aspect, there is provided herein, a method of identifying an anti-multiple myeloma (MM) related disease agent, comprising: providing a test agent to a cell, and measuring the level of at least one miR gene product associated with altered expression levels in MM cells, wherein the alteration in the level of the miR gene product in the cell, relative to a suitable control cell, is indicative of the test agent being an anti-cancer agent.

In another broad aspect, there is provided herein, a marker for assessing one or more metabolic pathways that contribute to at least one of initiation, progression, severity, pathology, aggressiveness, grade, activity, disability, mortality, morbidity, disease sub-classification or other underlying pathogenic or pathological feature of a multiple myeloma (MM) related disease, wherein the marker comprises one or more gene products coding for least one isolated miR gene product is miR-21, miR-25, miR-106b˜25 cluster, miR-181a, miR-181b, miR-106a, miR-17˜92 cluster, miR-19a, miR-19b and miR-32. In certain embodiments, the at least one miR gene product is one or more of: miR-21, miR-19a, miR-19b, miR-181a, miR-181b and miR-32. In certain embodiments, the at least one miR gene product is one or more of miR-19a and miR-19b.

In another broad aspect, there is provided herein, an article of manufacture comprising: at least one capture reagent that binds to a marker for a multiple myeloma related disease selected from at least one of the marker as described herein.

In another broad aspect, there is provided herein, a reagent for testing for a multiple myeloma (MM) related disease, wherein the reagent comprises an antibody that recognizes a protein encoded by at least one marker as described herein.

In another broad aspect, there is provided herein, a method of assessing the effectiveness of a therapy to prevent, diagnose and/or treat multiple myeloma (MM) comprising: 1) subjecting a subject to a therapy whose effectiveness is being assessed, and 2) determining the level of effectiveness of the treatment being tested in treating or preventing multiple myeloma (MM) by evaluating at least one marker as described herein. In certain embodiments, the candidate therapeutic agent comprises one or more of: pharmaceutical compositions, nutraceutical compositions, and homeopathic compositions. In certain embodiments, the therapy being assessed is for use in a human subject.

In another broad aspect, there is provided herein, a kit for screening for a candidate compound for a therapeutic agent to treat a multiple myeloma (MM) related disease, wherein the kit comprises: one or more reagents of at least one marker as described herein, and a cell expressing at least one marker. In certain embodiments, the presence of the marker is detected using a reagent comprising an antibody or an antibody fragment which specifically binds with at least one marker.

In another broad aspect, there is provided herein a screening test for multiple myeloma (MM) comprising: contacting one or more of the markers described herein with a substrate for such marker and with a test agent; and, determining whether the test agent modulates the activity of the marker. In certain embodiments, all method steps are performed in vitro.

In another broad aspect, there is provided herein, a method for treating, preventing, reversing or limiting the severity of a multiple myeloma (MM) complication in an individual in need thereof, comprising: administering to the individual an agent that interferes with at multiple myeloma (MM) response signaling pathway, in an amount sufficient to interfere with such signaling, wherein the agent comprises at least one miR gene product that interferes with SOCS-1 expression.

In another broad aspect, there is provided herein, use of an agent that interferes with at a multiple myeloma (MM) response signaling pathway, for the manufacture of a medicament for treating, preventing, reversing or limiting the severity of a multiple myeloma (MM) complication in an individual, wherein the agent comprises at least one gene product selected from a group consisting at least one miR gene product of miR-21, miR-106b˜25 cluster, miR-181a, miR-181b, miR-17˜92 cluster, miR-19a, miR-19b, and miR-32 and combinations thereof.

In another broad aspect, there is provided herein, a pharmaceutical composition for treating a multiple myeloma (MM) cancer, comprising at least one p300-CBP associated factor expression-inhibition compound, and a pharmaceutically-acceptable carrier.

In another broad aspect, there is provided herein, a method for controlling p53 activity in a cell in need thereof, comprising contacting the cell with at least one miR gene product in an amount sufficient to control such activity, wherein the miR gene product is one or more of: miR-106b-25 cluster, miR-32, miR-181a and miR-181b. In certain embodiments, the miR gene product targets the p300-CBP-associated factor (PCAF) gene. In certain embodiments, the cell is a multiple myeloma (MM) cell.

In another broad aspect, there is provided herein, an miRNA signature associated with a MM multi step transformation process from normal to via MGUS to clinically overt MM, comprising: at least one or more of: miR-21, miR-25, miR-106b˜25 cluster, miR-181a, miR-181b, miR-106a, miR-17˜92 cluster, miR-19a, miR-19b and miR-32. In certain embodiments, the at least one miR gene product is one or more of: miR-21, miR-19a, miR-19b, miR-181a, miR-181b and miR-32. In certain embodiments, the at least one miR gene product is one or more of miR-19a and miR-19b.

In another broad aspect, there is provided herein, a method for blocking apoptosis and/or promoting cell survival in a subject in need thereof, comprising administering an effective amount of one or more miR gene products, wherein the gene product is one or more of: miR-17˜92, miR-19a, miR-19b and miR-21.

In another broad aspect, there is provided herein, a method for targeting PCAF, a p53 positive regulator in a subject in need thereof, comprising administering an effective amount of one or more miR gene products, wherein the miR gene product is one or more of: miR106b˜25, miR-181a and miR-32.

In another broad aspect, there is provided herein, a method for downregulating SOCS-1 and/or activating IL-6 in later stages in MM pathogenesis in a cell, comprising up-regulating miR-19s in the cell.

In another broad aspect, there is provided herein use of both miRNA microarrays and quantitative RT-PCR to assess the miRNA expression in MM-derived cell lines and CD138+ bone marrow plasma cells (PCs) from MM patients, MGUS and normal donors.

Also provided herein is a miRNA signature related to expression and regulation of proteins associated with malignant transformation of PCs.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference.

### EXAMPLES

A Characteristic miRNA Signature Differentiates MGUS from Healthy PCs

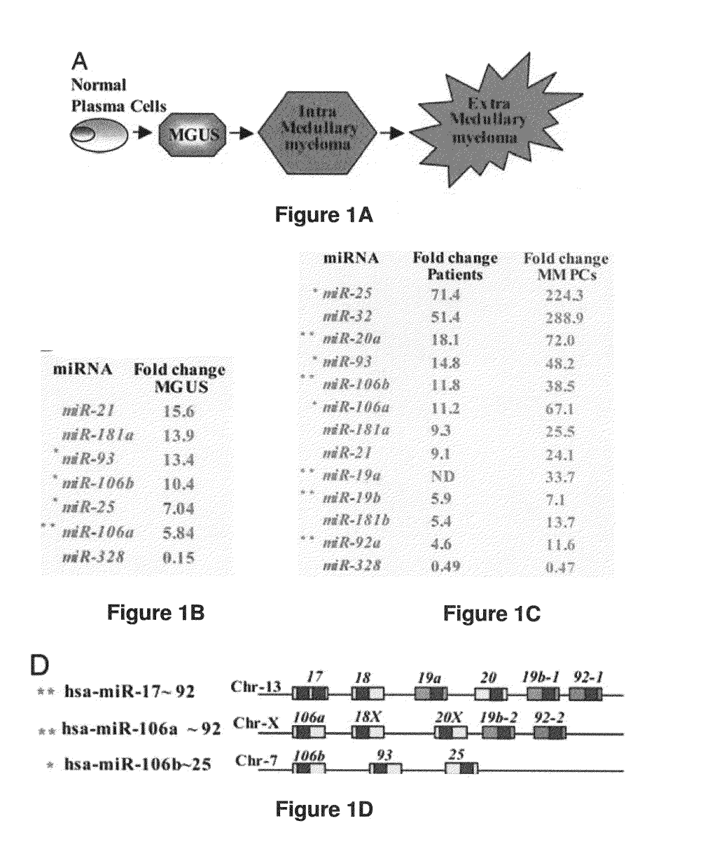

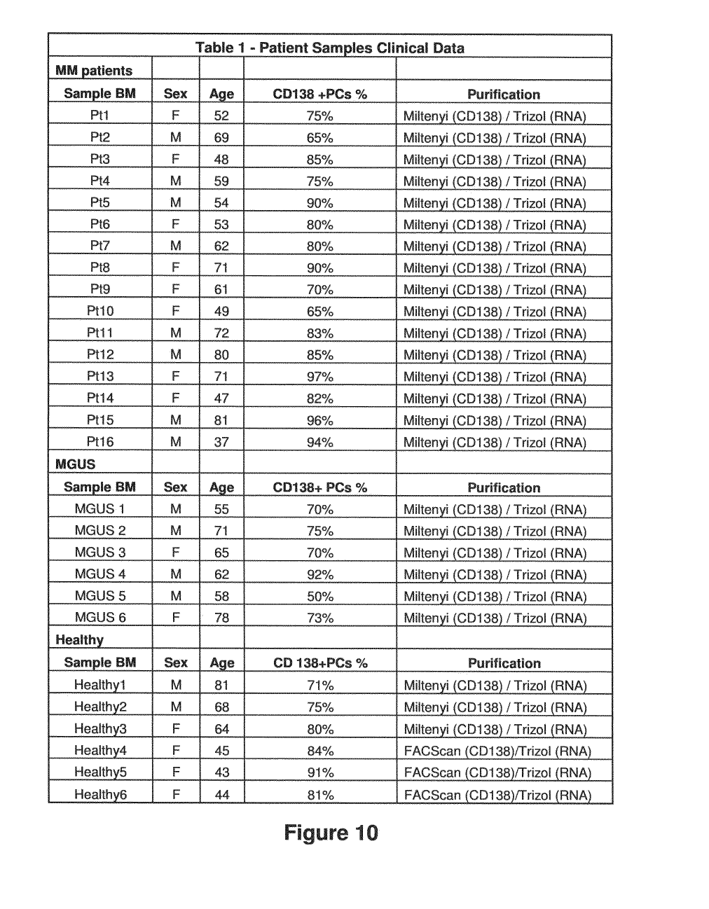

Current models assume that MM evolves through a multi step transformation process (FIG. 1A) (15). To identify specific alterations associated with early pathogenetic events of MM, the inventor profiled 5 CD138+ PCs from MGUS subjects and 4 healthy PCs [see FIG. 10—Table 1 for patient characteristics] by using the miRNA microarray platform described herein.

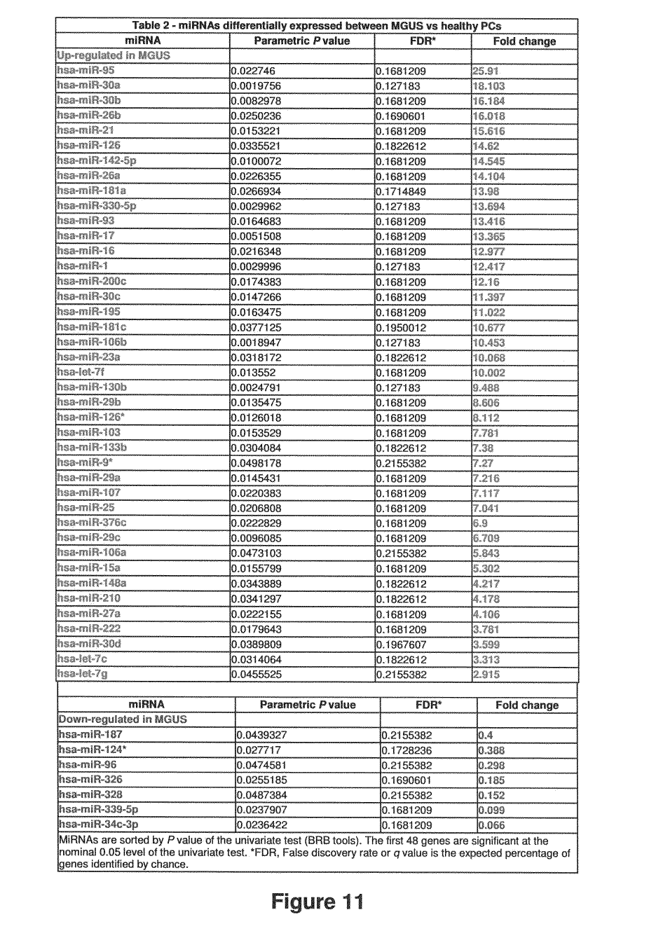

We first compared MGUS to the healthy counterpart PCs by using the univariate t test within BRB tools (Class comparison) (FIG. 11—Table 2, FIG. 1B). The inventor found 48 miRNAs significantly deregulated (p-value≦0.05); 41 miRNAs were upregulated and 7 downregulated in MGUS with respect to normal CD138+ PCs (FIG. 11—Table 2, a representative list is shown FIG. 1B).

The most upregulated miRNAs in MGUS were miR-21 which was described to be upregulated in MM as well (14), miR-181a, known to have a role in B and T cell differentiation (16) and the oncogenic cluster miR-106b˜25, in particular miR-93, miR106b and miR-25 (FIG. 11—Table 2, FIG. 1B).

MiRNA Signatures in MM Patients and Cell Lines

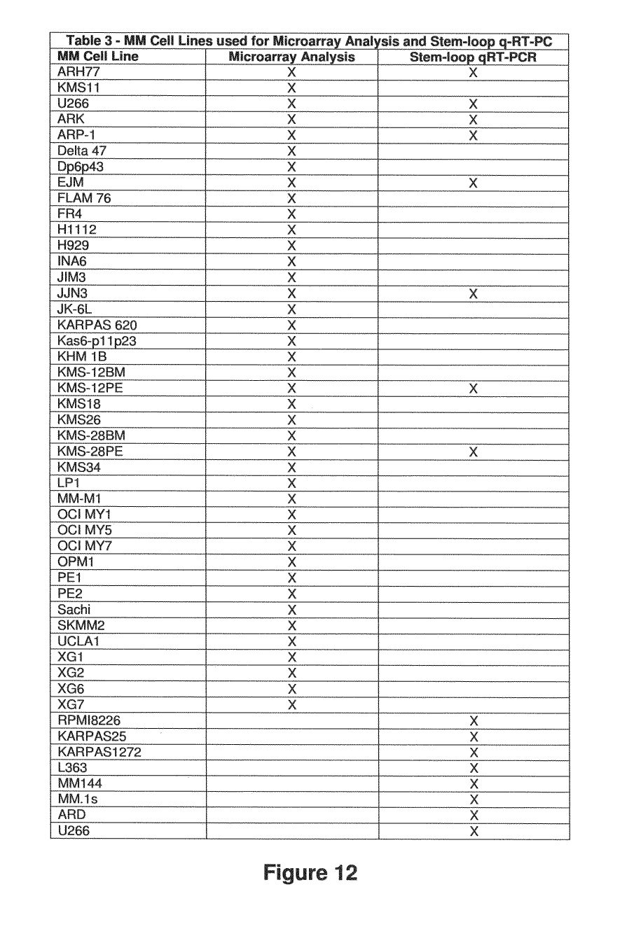

To determine whether miRNAs are deregulated in MM, the inventor analyzed the global miRNA expression in 41 MM-derived cell lines (FIG. 12—Table 3), CD138+ untreated bone marrow PCs from 10 MM and 4 normal CD138+ PCs using the inventor's miRNA microarray (17). The degree of CD138+PCs purity after AutoMACs automated separation system (Miltenyi-Biotec, Auburn, Calif.) and the clinical features of the MM cases and normal PCs are listed in FIG. 10—Table 1.

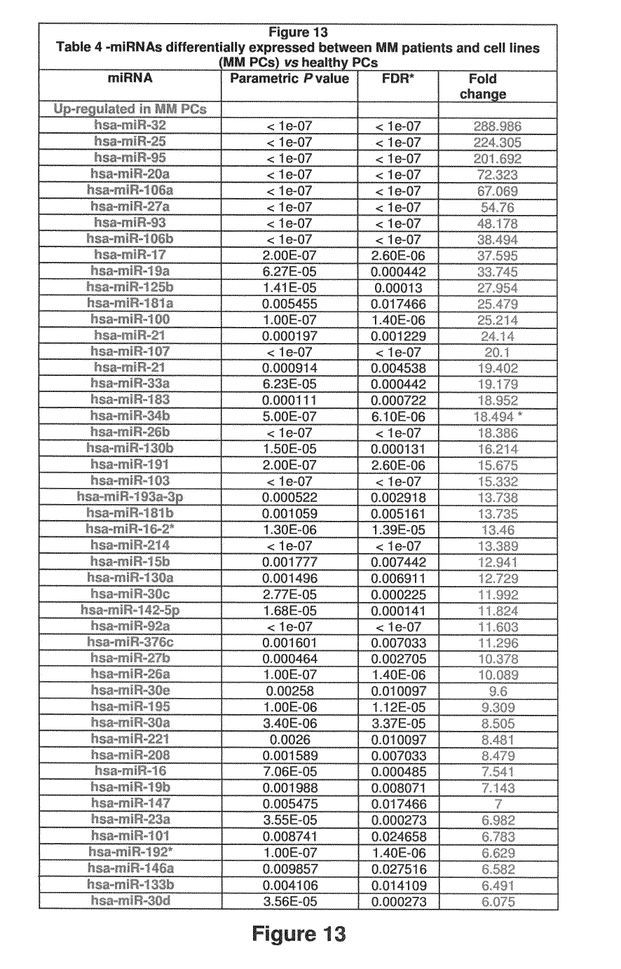

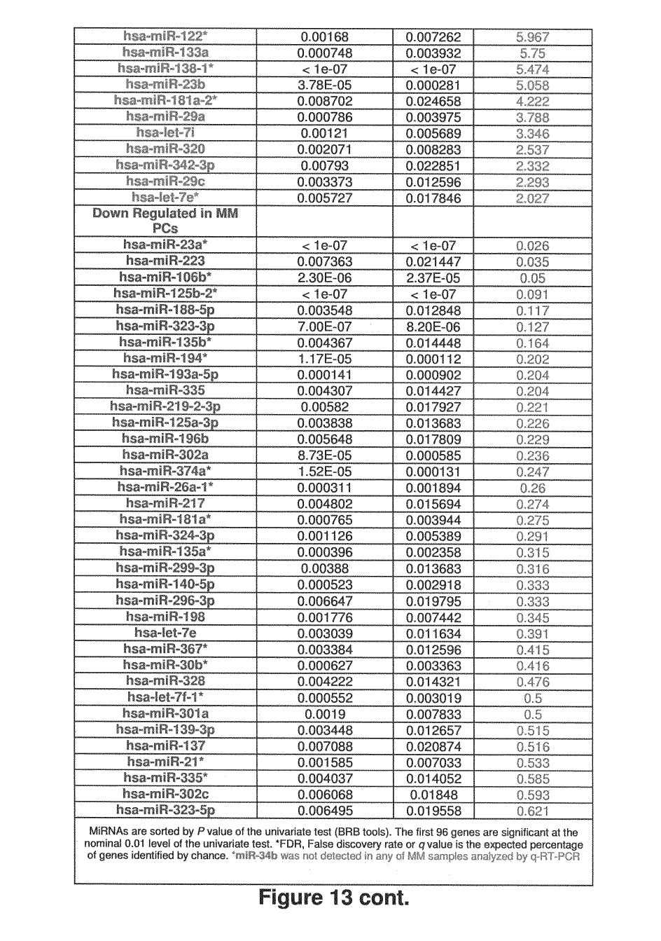

First, the inventor compared miRNA expression in primary tumors and cell lines compared to CD138+ healthy controls using univariate t-test within by BRB (FIG. 13—Table 4). The inventor's analysis revealed upregulation of 60 and downregulation of 36 miRNAs in MM patients and cell lines compared to CD138+ healthy controls (FIG. 13—Table 4, FIG. 1C).

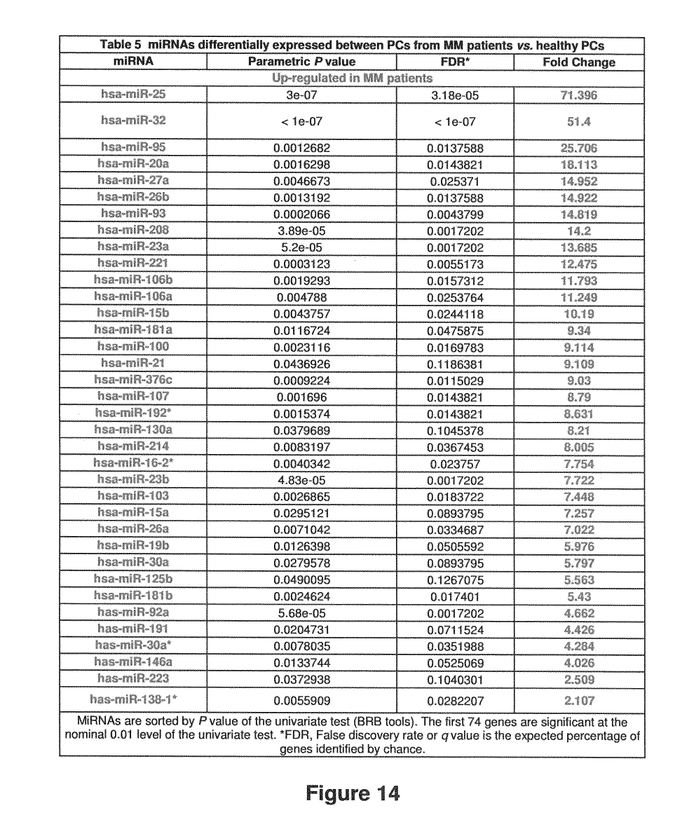

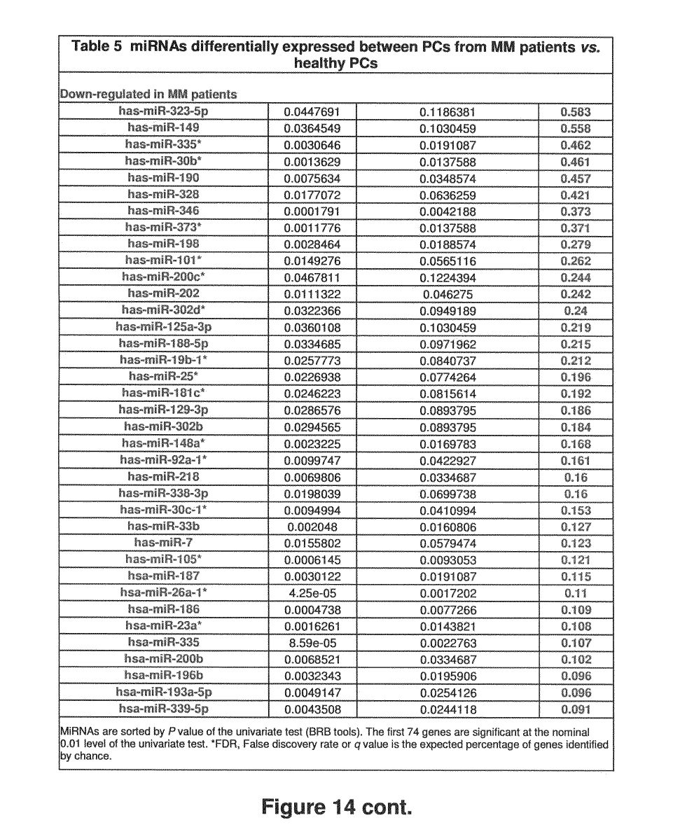

All miRNAs have a fold change more than 2 and a p-value≦0.01. Since miRNA expression in cell lines could be also de-regulated due to prolonged in vitro culture, the inventor analyzed the miRNA expression only in MM patients versus healthy PCs (FIG. 14—Table 5).

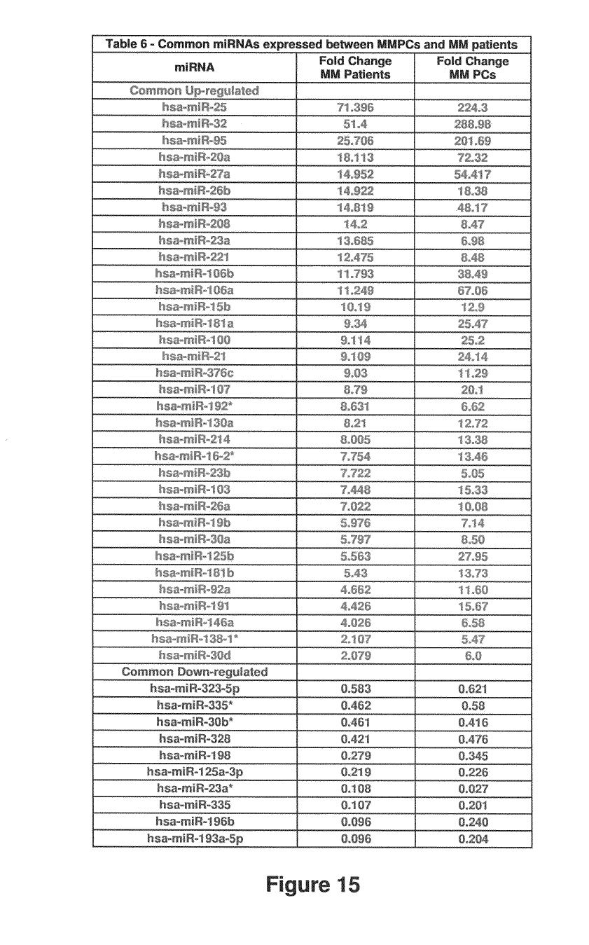

The inventor found 37 up and 37 downregulated miRNAs in MM patients with respect to normal PCs with a fold change >2 and a p-value≦0.01 (FIG. 14—Table 5). About 90% of the upregulated miRNAs (34 out of 37) and 30% of the downregulated miRNAs (10 out of 37) were in common to the combined group of MM patients and cell lines, thereby validating the inventor's approach of combining cell lines and MM patient samples, at least for the analysis of up-regulated miRNAs (FIG. 1C) (FIG. 15—Table 6).

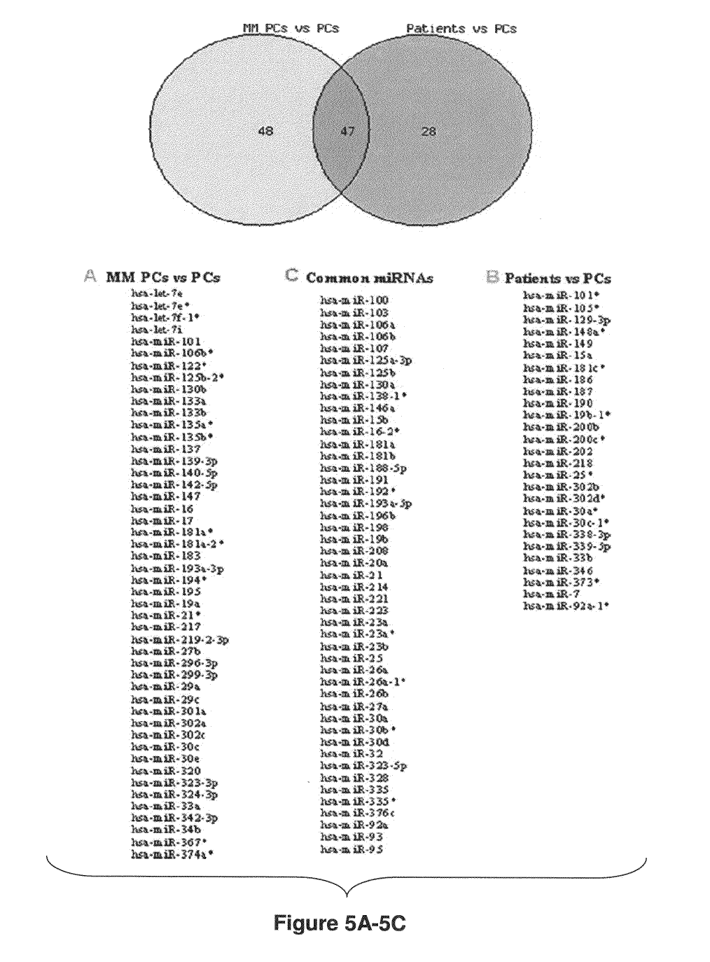

A Venn diagram in FIG. 5 shows the common miRNAs between these two groups of comparison. Similar to the signature observed in MGUS, miR-21 and the miR-106a˜92 cluster, were found upregulated in MM patients and cell lines (FIG. 1C, FIG. 12—Table 3). However, the inventor identified that miR-32 and the cluster miR-17˜92, in particular miR-19a and miR-19b, were significantly upregulated only in MM samples but not in MGUS or healthy PCs (FIG. 1C), indicating a possible role in the malignant transformation from MGUS to MM.

Validation of the miRNA Signatures by q-RT-PCR

To validate the microarray results the inventor performed q-RT-PCR for miR-32, miR-17-5, miR-19a, miR-19b, miR-20a, miR-92, miR-106a (miR-17˜92 cluster), miR-106b, miR-93 and miR-25 (miR-106b˜25 cluster), miR-328 and miR-181a and miR-181b using an independent set of randomly chosen CD138+PCs from healthy subjects (n=3), MM patient samples (n=6) and MGUS (n=3) (FIG. 10—Table 1) all from different donors, plus a set of MM cell lines (n=15) (FIG. 10—Table 1).

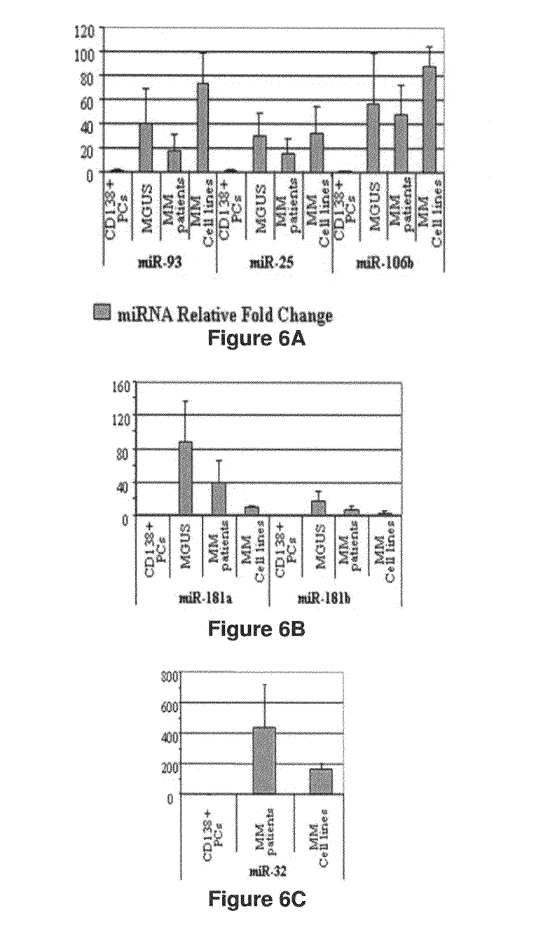

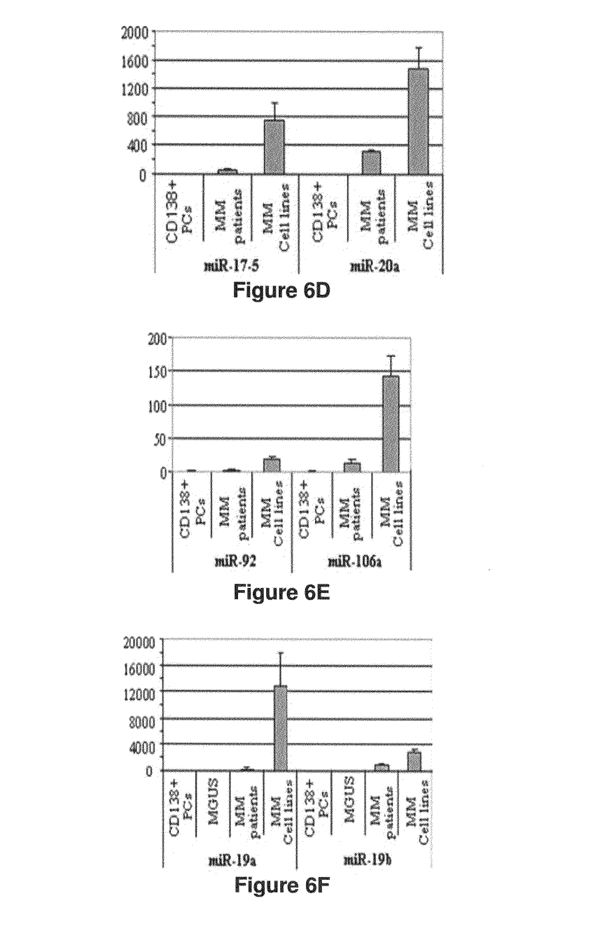

The inventor confirmed the over-expression of the miR-106b˜25 cluster in MGUS and MM samples with respect to the CD138+ healthy PCs (FIG. 6A). Although the miR-106b˜25 cluster shares a high degree of homology with the miR-17˜92 cluster (FIG. 1D), and an oncogenic role was reported for both (18-20), the inventor is confident of the specificity of stem-loop q-RT-PCR for the analysis of highly similar miRNAs; a previous report from the inventor's laboratory showed exquisite specificity of miR-106b, miR-93, and miR-25 primers (20). Mature miR-181a was over-expressed in 2/3 MGUS, 6/6 MMs, and 9/15 cell lines with an average on the differential expression shown in FIG. 6B.

In addition, miR-181b was also overexpressed in MM and MGUS, albeit at a lower degree than miR-181a (FIG. 6B). The inventor further validated the overexpression of the miR-32 and miR-17˜92 cluster (FIG. 6C-F) in MM patients and cell lines. Consistent with the array data, the two miR-17˜92 cluster members, miR-19a and miR-19b, and miR-32 were highly over-expressed in 6/6 MM PC samples and 15/15 cell lines (FIGS. 6C, 6F). Principally the inventor found that miR-19a and miR-19b have a fold change >100 times (FIG. 6F), though they show very low expression in 1/3 MGUS and almost no expression in 2/3 MGUS and 3/3 healthy PCs samples, validating the inventor's initial arrays results and suggesting that these miRNAs are MM specific.

Several miRNAs Upregulated in MM Target PCAF, a Positive Regulator of p53

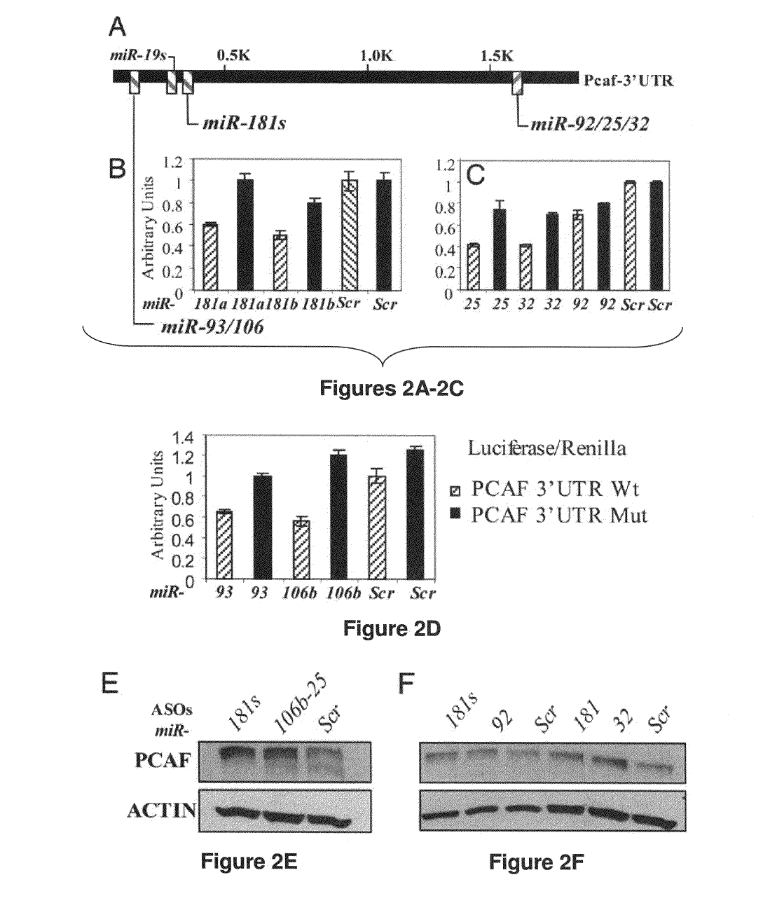

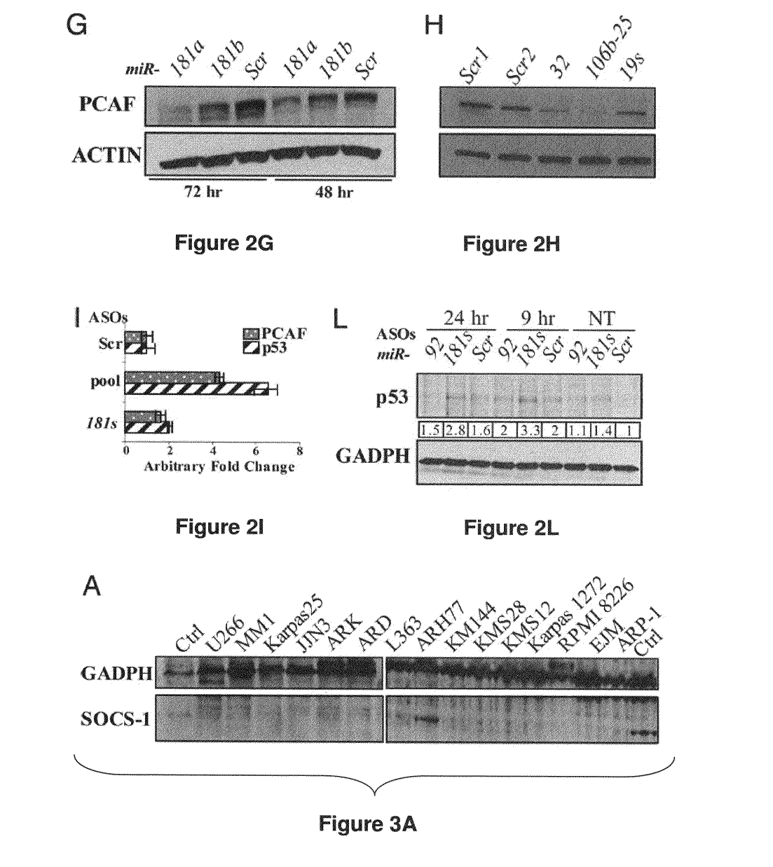

One of the most upregulated miRNAs in MGUS and MM patients and cell lines, were miR-181a and miR-181b, and miR-106b˜25, while miR-32 was preferentially upregulated in MM. Using “in silico” target prediction software [Target Scan (21), Pictar (22)], the inventor found that those miRNAs are predicted to target the 3′-UTR of the p300-CBP-associated factor (PCAF) (FIG. 2A). PCAF is a histone acetyltransferase (HAT) involved in the reversible acetylation of various transcriptional regulators, including the tumor suppressor protein p53 (23). Recently Linares et al. have shown that PCAF possesses an intrinsic ubiquitination activity that is critical for controlling Hdm2 expression levels and thus p53 (24) that is rarely mutated (5-10% of cases) or deleted at diagnosis in MM (25-26).

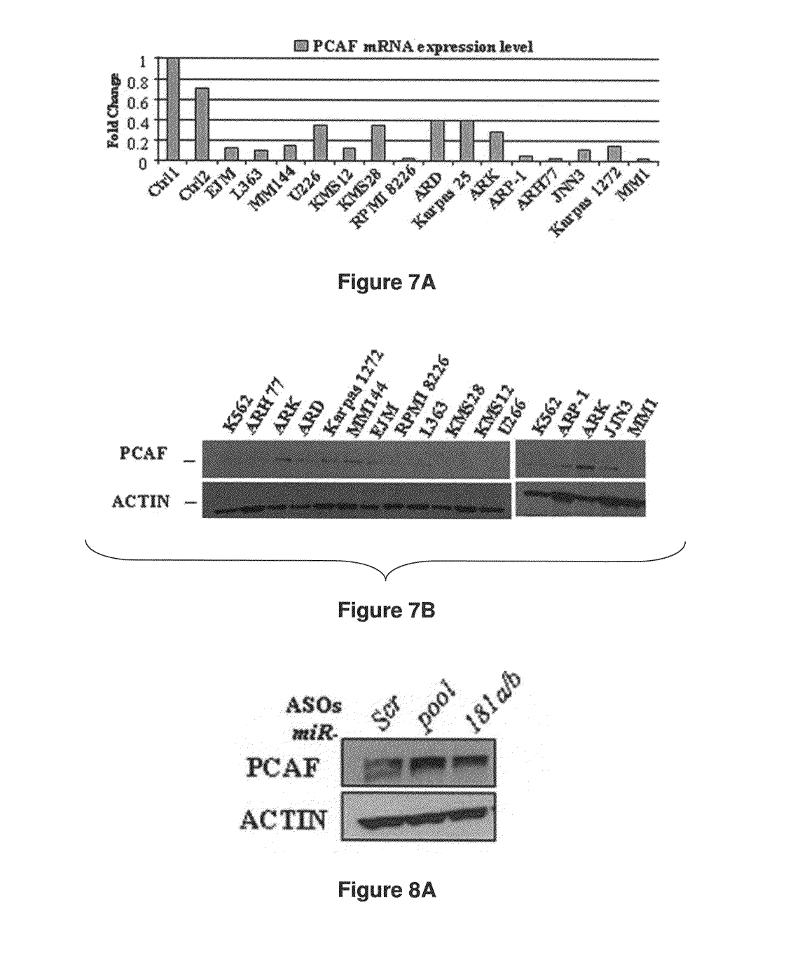

To examine whether these miRNAs could regulate PCAF, first the inventor analyzed PCAF expression by q-RT-PCR (FIG. 7A) and Western blotting (FIG. 7B) in 15 MM cell lines. As control, the inventor used two CD138+PCs isolated from healthy donors. The inventor found that PCAF expression is almost absent (10 fold less than in control) in 10/15 cell lines whereas the remaining 5 cell lines displayed very low expression. To investigate whether this gene was deleted at the genomic level the inventor performed whole genome CGH analysis of all fifteen MM cell lines using the Affymetrix SNP 6.0 arrays. However, the inventor did not observe deletion of the PCAF gene (data not shown).

Second, the inventor cloned the PCAF 3′ UTR 5 into a luciferase reporter vector and co-transfected with the candidate miRNAs mimics or scrambled oligonucleotides and performed luciferase assays as described in methods. The inventor found that miR-181a/b (FIG. 2B), miR-106b˜25 cluster and miR-32 (FIGS. 2D, 2B), interact with the PCAF 3′UTR in vitro. However, this interaction was less significant with miR-92 (FIG. 2C) and no interaction was observed with miR-19a and miR-19b (data not shown). Mutation of the predicted miRNA binding sites in the reporter vector abrogated this effect, indicating that these miRNAs directly interact with the PCAF 3′ UTR.

To confirm the biological role of these miRNAs in PCAF regulation in MM cells, the inventor validated the in vitro studies, by antagonizing endogenous miR-181a, miR-181b, miR-25, miR-93, miR-106b and MiR-92 using anti-sense oligos (ASOs) in U266 and JJN3 MM cell lines. In both cell lines the antagomiRs induced accumulation of PCAF protein expression at 72 hrs after transfection (FIGS. 2E, 2F). By contrast, overexpression of the same miRNAs by oligonucleotide transfection reduced PCAF expression in K562 cell line (FIGS. 2G, 2H). MiR-19a and miR-19b did not influence PCAF expression (FIG. 2H) and miR-92 had little effect on its expression (FIG. 2F), confirming the luciferase expression data (FIG. 2C).

To determine whether the miRNAs regulators of PCAF are able to indirectly affect p53 expression, the inventor transfected MM1 cells with anti-miR-181a/b or with all antagomiRs simultaneously (anti-miRs-181s, anti-miR-93, anti-miR-106b, anti-miR-25, anti-miR-32) exposed the cells to ultraviolet (UV) radiation (FIG. 2I), and measured the expression of p53 and PCAF by qRT-PCR (FIG. 2I).

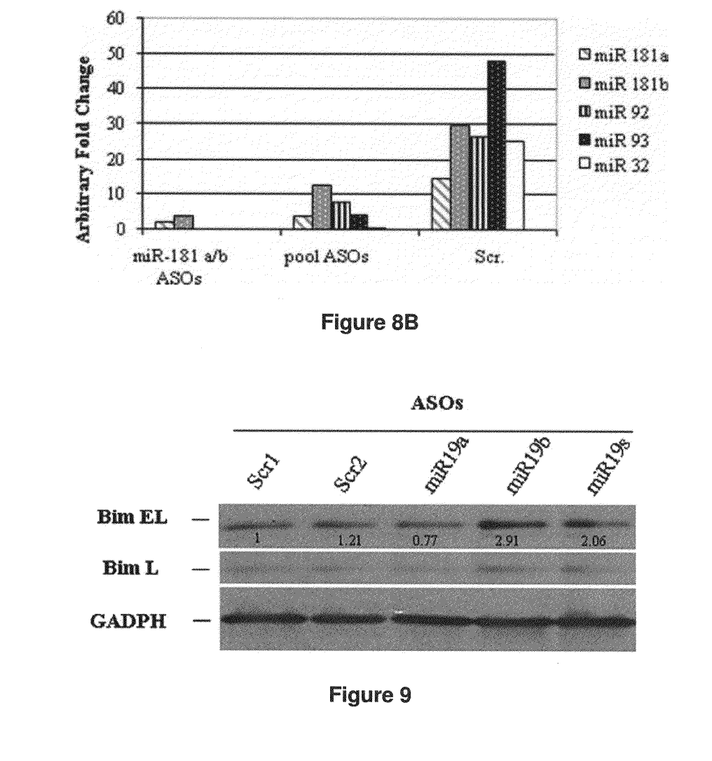

FIG. 8A shows the re-expression of PCAF protein after 48 hr of antagomiRs treatment. The antagonizing activity of transfectant oligos is shown in FIG. 8B. After UV treatment, p53 mRNA expression was almost doubled in cells transfected with antisense miR-181a/b, while it increased 6-fold after nucleoporation with all antagomiRs simultaneously (FIG. 2I). Furthermore, after transfection of antisense miR-181a/b oligos in MM.1s, the inventor treated the cells with a small-molecule MDM2 antagonist nutlin-3a (10 μM) and measured p53 by Western Blotting. The inventor found that the cells transfected with miR-181s anti-sense oligos (ASOs) displayed a higher level of p3 protein compared to scrambled and miR-29 AOSs at 9 and 12 hrs (FIG. 2L). Together these data show that the miR-106b-25 cluster, miR-32, miR-181a and miR-181b target PCAF and through this gene, control indirectly p53 activity in myeloma.

miR-19a and miR-19b Target SOCS-1, a Negative Regulator of IL-6R/STAT3 Pathway

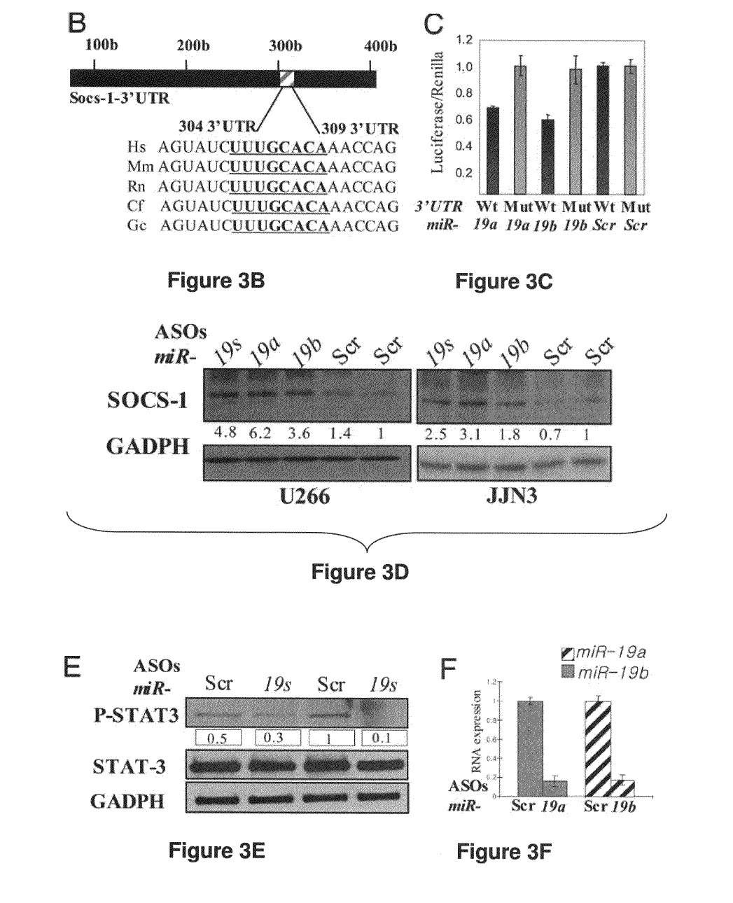

Our findings indicate that miR-19a and miR-19b are upregulated >100-fold in patient samples, >2000 times in the cell lines (FIG. 6F) and are both almost absent in normal PCs and MGUS. Both miRNAs contribute to the development of MM. Therefore, the inventor searched for miR-19a and b mRNA targets, which are involved in myeloma pathogenesis, using available target prediction software [Target Scan (21), Pictar (22)]. Among more than 100 predicted targets, SOCS-1 has been implicated in the negative regulation of several cytokine pathways including IL-6, particularly the Jak/STAT pathways (27) and is frequently silenced by methylation in MM (28). The inventor now believes that the high levels of miR-19s levels in MM samples may play an important role in the constitutive activation of Jak/STAT-3 signaling through down regulation of its negative regulator SOCS-1. The inventor first assessed its expression in 15 MM cell lines and two healthy CD138+ PCs and found almost no protein expression in 13/15 cell lines compared to control (FIG. 3A).

To examine whether SOCS-1 reduced expression levels could be a consequences of upregulated miR-19a/b in MM cells, the inventor performed Western blot analysis using SOCS-1 antibody after transfection of candidate antagomiRNAs or scrambled oligonucleotides in U266 (which has an active IL-6 autocrine loop) (29) and JJN3 MM cell lines which display reduced SOCS-1 expression (FIG. 3D).

Moreover, miR-19a and miR-19b mimics inhibited the expression of a reporter vector containing SOCS-1 3′ UTR, while mutation of the predicted miRNA-binding site abrogated this effect (FIGS. 3B, 3C) after transfection by anti-miR-19s but not by scrambled (FIG. 3E).

The ASOs activity at 72 hrs was detected by q-RT-PCR (FIG. 3F). These studies show a role of miR-19s in the IL-6 anti-apoptotic signal in the pathogenesis and malignant growth of MM.

miR-17˜92 Cluster Target Bim in MM Cells

Since the miR-17˜92 cluster has been shown to target the proapoptotic gene, Bim (19-20), the inventor examined whether Bim expression is modulated by miR-17-92 in MM cells. U266 cells were transfected with miR-19s ASOs and Bim expression was evaluated using immunoblot. The inventor found a significant increase of Bim protein levels at 48 hr following treatment with anti-miR-19s compared to scrambled oligonucleotides (FIG. 9). Together, these results have supported previous published data (19-20) that show Bim as direct target of miR-17˜92 and suggest a possible mechanism through which overexpression of miR-17˜92 contributes to the antiapoptotic signals in MM.

Ectopic Repression of miR-19s and miR-181a/b in MM Cell Lines Leads to Significant Suppression of Tumor Growth in Nude Mice

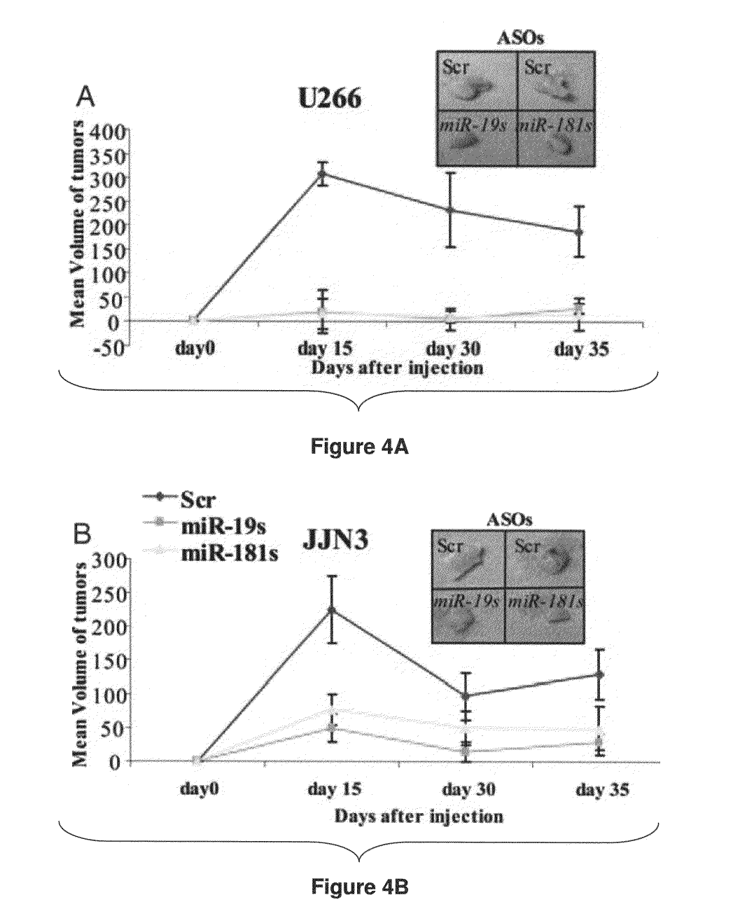

To explore the in vivo relevance of the inventor's observations, the inventor examined the tumorigenicity of U266 and JJN3 cells in athymic nu/nu mice following silencing of the endogenous miR-19s and miR-181a/b. Two independent experiments, each using 16 mice for each cell line were conducted. U266 or JJN3 cells were transfected with ASOs or scrambled oligonucleotides in vitro. The inventor confirmed transfection efficiency (80% for U266 and JJN3) using BLOCK-IT Fluorescent Oligo (Invitrogen). Twenty-four hours later 3×10⁷ viable cells for each group, re-suspended in 100 μl of BD matrigel matrix, were oligonucleotide developed measurable tumors. By contrast, mice transplanted with cells expressing antagomiRs showed significant inhibition of tumor growth compared with controls (P<0.01) (FIGS. 4A, 4B).

Both cell lines treated with anti-miR-19s showed tumor volumes >10-fold reduced and tumors treated with antagomiR-181s were three times smaller in JJN3 cells and 10 times smaller in U266 cells, P=0.02 and P=0.01, respectively.

Importantly, complete tumor suppression was observed in two mice injected with U266 cells treated with anti miR-19s. The average tumor volume after four weeks for U266 cells was 308.5 mm³ and for JJN3 cells was 225 mm³. Only 50% of mice injected with MM cells transfected with the antago miR-19s and miR-181s developed measurable tumors at 4 wks. The average tumor volume for U266/anti miR-19s was 19.5 mm³ and for U266/anti miR-181s was 14 mm³. Similar results were observed with JJN3 MM cells, with average tumor volumes of 25 mm3 and 80 mm³ for anti miR-19s and anti-miR-181s, respectively (FIGS. 4A, 4B). Taken together these results show an oncogenic role of these microRNAs in MM and the inventor herein now believes that the stronger effect of anti miR-19s is related to the IL-6 dependence of U266.

Discussion

Over the past few years several studies have illustrated the biological relevance of miRNAs for the differentiation of normal hematopoietic cells and the contribution of deregulated miRNA expression in malignant counterparts (11,30,31).

The inventor herein now shows the first comprehensive global microRNAs expression profiling of MM, MGUS and contrasted these expression patterns with that of normal PCs. The similar miRNA expression pattern observed in MM cell lines and in primary newly diagnosed MMs supports the research design as described herein. In addition, previous MM microarrays studies have combined MM cell lines with primary patient samples, validating the inventor's strategy (32).

Further, the inventor now identifies a miRNA signature that can be associated with a MM multi step transformation process from normal PCs via MGUS to clinically overt myeloma, while still being conscious of limitations due to the low number of MGUS and primary tumor samples.

In MGUS patients, the inventor identified upregulated miRNAs with oncogenic function, such as the miR-21 and the miR-106b˜25 cluster. MiR-21 is upregulated in many solid and hematological tumors (11,25). The ectopic expression of miR-21 in glioblastoma cells blocks apoptosis (33), while silencing its expression in several cancer cells, inhibits cell growth and leads to increased apoptotic cell death by unblocking the expression of its targets; tumor suppressor genes like phosphatase and tensin homolog (PTEN) and protein Programmed Cell Death 4 (PDCD4) (34). Petrocca et al. (20) have shown that the miR-106b˜25 cluster play a role in gastric cancer tumorigenesis by targeting the pro-apoptotic Bim and p21. Thus, these two miRNA may contribute to earlier steps in plasma cell transformation by blocking apoptosis, promoting PCs survival and predisposing to secondary genetics abnormalities that will ultimately result in a full blown malignancy.

In MM (including MM cell lines and primary tumors versus normal PCs) the inventor identified a signature comprised of multiple upregulated miRNAs, including among others; miR-32, miR-21, miR-17˜92, miR-106˜25, and miR-181a and miR-181b.

While miR-106˜25, miR-181a and b and miR-21 were upregulated also in MGUS patients with respect to normal PCs, miR-32 and the miR-17˜92 cluster were highly expressed only in MM patients, suggesting that these miRNAs are MM specific.

Besides RAS mutations, no other genetic abnormality has been found to differentiate MGUS from MM (3). Thereby, miR-32 and the miR-17˜92 cluster may represent MM specific genetic changes.

Similar to the miR-106˜25 cluster, the oncogenic role of the miR-17˜92 cluster in B cell lymphoma is well known and several known proapoptotic genes, including PTEN, E2F1, and Bcl2111/Bim are confirmed as targets of miR-17˜92 (35-36). Recently Ventura et al. (19) have shown that the miR-17˜92 cluster is also essential for B cell development and that the absence of miR-17˜92 leads to increased levels of the proapoptotic protein Bim and inhibits B cell development at the pro-B to pre-B transition. However, given the nearly identical sequences, it is very likely that miR-106b˜25 and miR-17˜92 clusters cooperate in exerting similar, if not identical, functions as in targeting Bim (19-20).

Now shown herein are important functional insights about miRNAs deregulated in MM. The inventor has confirmed that the proapoptotic Bim is a target of the miR-17˜92 cluster in MM cells. Therefore, miR-17˜92 along with miR-21 blocks apoptosis and promotes cell survival. On the other hand, miR106b˜25, miR-181a and miR-32 [but not miR17˜92 cluster (specifically miR-19s and miR-92)], target PCAF, a p53 positive regulator.

While not wishing to be bound by theory, the inventor herein now believes that, consistent with the low frequency of p53 mutations in MM, the down regulation of PCAF by miR-106b˜25 cluster, miR-181s and miR-32 keeps p53 at low level or partially inactivated, by controlling its stability through Hdm2 (24) and working as a histone acetyltransferase (HAT) (23).

Also first described herein is the specific role of miR-19s on the STAT3/IL6R negative regulator SOCS-1. In fact, IL-6 pathways in MM are among the best characterized survival pathways participating in PC transformation and oncogenesis through STAT3, impacting apoptosis regulators such as the Bcl-2 family members (1, 16). These findings demonstrate that the up-regulation of miR-19s in MM can contribute to SOCS-1 downregulation and IL-6 activation at later stages in MM pathogenesis.

The role of miR-19s and miR-181s in MM cells as oncomiRNAs was confirmed by in vivo studies. The data demonstrate significant tumor regression of transplanted tumors following treatment with miR-19s and 181s antagomirs. These data now show that miRNAs can have a therapeutic potential in antagonizing the growth of transformed PCs.

In conclusion, described herein are distinctive miRNA signatures in MM and MGUS, characterized by overexpression of miRNAs with known oncogenic activity. The data provide insights into the miRNA function in MM by establishing links with the regulation of critical pathways in MM by miRNAs, including apoptosis, survival and proliferation. These results indicate an additional level of control by this class of regulatory molecules in the multi step process associated with malignant transformation of PCs.

Material and Methods

RNA extraction and miRNA microarray experiments RNA extraction and miRNA microchip experiments were performed as described in detail elsewhere (37). The miRNA microarray is based on a one-channel system (35). Five micrograms of total RNA was used for hybridization on the OSU custom miRNA microarray chips (OSU_CCC version 3.0), which contains x1,100 miRNA probes, including 345 human and 249 mouse miRNA genes, spotted in duplicates.

RT-PCR The single tube TaqMan miRNA assays were used to detect and quantify mature miRNAs as previously described (10) using ABI Prism 7900HT sequence detection systems (Applied Biosystems). Normalization was performed with RNU6B. Comparative real-time PCR was performed in triplicate, including no-template controls. Relative expression was calculated using the comparative Ct method.

ASOs and Mimics transfection experiments Cells were transfected by using nucleoporation (Amaxa) kit V (for JJN3 and MM1s cell lines) and kit C (for U266 cell line) using 100 nM miRNA precursors (Ambion), or 100 nM LNA miRNA antisense oligonucleotides (Ambion). Protein lysates and total RNA were collected at the time indicated. miRNA processing and expression were verified by northern blot and stem-loop qRT-PCR. The inventor confirmed transfection efficiency (≈80% for U266 and JJN3 and 50% for MM1s) using BLOCK-IT Fluorescent Oligo (Invitrogen) for all the cell lines. Untreated cells transfected with negative control oligonucleotides were used as a calibrator.

Cell collection and total RNA purification Samples included PCs from 16 newly diagnosed cases of MM, 6 patients with monoclonal gammopathy of undetermined significance (MGUS), and 6 healthy donors (normal PCs). Written informed consent was obtained in keeping with institutional policies. PC isolation from mononuclear cell fraction was performed by immunomagnetic bead selection with monoclonal mouse antihuman CD138 antibodies using the AutoMACs automated separation system (Miltenyi-Biotec, Auburn, Calif.). PC purity is shown in FIG. 11—Table 2, immunocytochemistry for cytoplasmic light-chain immunoglobulin (Ig), and morphology by Wright-Giemsa staining. Three of the six healthy PCs were obtained from ALLCELLS, LLC and the purity of PCs was assessed by FACScan Analysis and was more than 80%. MM cell lines (FIG. 12—Table 3) [courtesy of Dr M. Kuehl (National Cancer Institute, MD), Dr. Joshua Epstein (Little Rock, Ark.), Dr S. Rosen (Chicago, Ill.), Dr. M. Gramatzki (Kiel, Germany)] and an Epstein-Barr virus (EBV)-transformed B-lymphoblastoid cell line (ARH-77, ARK, UCLA-1) were grown as recommended (American Type Culture Collection, Chantilly, Va.). Total RNA was isolated with Trizol extraction reagent (Invitrogen).

Luciferase Reporter Vectors PCAF and SOCS1 3′UTR containing predicted microRNA binding site were amplified by PCR from genomic DNA (293T/17cells) and inserted into pGL3 control vector (Promega) by using Xbal site immediately downstream from the stop codon of firefly luciferase. Deletion of the first six nucleotides of each complementary seed-region complementary site were inserted in mutant construct using quick change site directed mutagenesis kit (Stratagene), according to the manufacture's protocol. Primer sequences are available upon request.

Luciferase Assays QBI293 and Meg01 cells were cotransfected in six-well plates with 1 μg of pGL3 firefly luciferase reporter vector (see luciferase reporter vector method), 0.1 μg of the phRL-SV40 control vector (Promega), and 100 nM miRNA precursors (Ambion) using Lipofectamine 2000 (Invitrogen). Firefly and Renilla luciferase activities were measured consecutively by using the Dual Luciferase Assay (Promega) 24 hr after transfection. Each reporter plasmid was transfected at least twice (on different days) and each sample was assayed in triplicate.

Xenograft Model Studies were performed under an Institutional Animal Care and Use Committee (IACUC) approved protocol. Eight-week-old male athymic nu/nu mice (Charles River Laboratories, Wilmington, Mass.) were maintained in accordance with IACUC procedures and guidelines. 30×10⁶ U266 cells or JJN3 transfected cells were suspended in 0.10 ml of extracellular matrix gel (BD Biosciences) and the mixture was injected subcutaneously into the right and left flanks. Serial measurements of xenograft growth were performed, and tumor volume was estimated using the formula 4/3π (L*W*H/8).

Western Blotting Immunoblot analyses were performed as described (10), using: rabbit polyclonal antiSOCS-1 (Abcam); rabbit polyclonal antisera against Gadph, Bim, Stat-3, P-Stat-3 (Tyr705) (Cell Signaling); rabbit polyclonal PCAF and monoclonal p53 (Santa Cruz). Protein levels were normalized relative to β actin or/and Gapdh level, detected with appropriate antisera (Santa Cruz Biotechnology).

UV and Nutlin3a treatment MM.1s and U266 MM cell lines were transfected with miR-181s, miR-106b-25, miR-32 and miR-19s ASOs as described herein and at 24 hrs after transfection U266 cells were treated with 10 μM Nutlin3a (Cayman Chemical Company) and MM.1S were treated with 6 J/m² UV (Ultra LUM. Inc. Paramount, Calif.) and harvested at the time described herein.

Data Analysis Microarray images were analyzed by using GenePix Pro 6.0. Average values of the replicate spots of each miRNA were background-subtracted and subject to further analysis. MiRNAs were retained when present in at least 50% of samples and when at least 50% of the miRNA had fold change of more than 1.5 from the gene median. Absent calls were thresholded to 4.5 in log2 scale before normalization and statistical analysis. This level is the average minimum intensity level detected above background in miRNA chips experiments. Quantiles normalization was implemented using the Bioconductor package/function. Differentially expressed microRNAs were identified by using the univariate t test within the BRB tools version 3.5.0. Set with a significant univariately at alpha level equal to 0.01. This tool is designed to analyze data using the parametric test t/F tests, and random variance t/F tests. The criteria for inclusion of a gene in the gene list is either p-value less than a specified threshold value, or specified limits on the number of false discoveries or proportion of false discoveries. The later are controlled by use of multivariate permutation test.

Examples of Uses

In one aspect, the present invention provides methods for predicting survival of a subject with cancer. The prediction method is based upon the differential expression of a plurality of biomarkers in cancer cells. It was discovered that some biomarkers tend to be over-expressed in short-term cancer survivors, whereas other biomarkers tend to be over-expressed in long-term cancer survivors. The unique pattern of expression of these biomarkers in a sample of cells from a subject with cancer may be used to predict relative survival time, and ultimately the prognosis, for that subject.

Method for Predicting Survival of a Subject With Cancer

One aspect of the invention provides a method for predicting cancer survival. The method comprises determining the differential expression of a plurality of biomarkers (i.e., miRs) in a sample of cells from a subject with cancer. The biomarker expression signature of the cancer may be used to derive a risk score that is predictive of survival from that cancer. The score may indicate low risk, such that the subject may survive a long time (i.e., longer than 5 years), or the score may indicate high risk, such that the subject may not survive a long time (i.e., less than two years).

Survival-Related Biomarkers

Some of the biomarkers are over-expressed in long-term survivors and some of the biomarkers are over-expressed in short-term survivors. A biomarker may play a role in cancer metastasis by affecting cell adhesion, cell motility, or inflammation and immune responses. A biomarker may also be involved in apoptosis. A biomarker may play a role in transport mechanism. A biomarker may also be associated with survival in other types of cancer

Measuring Expression of a Plurality of Biomarkers

One includes entails measuring the differential expression of a plurality of survival-related biomarkers in a sample of cells from a subject with cancer. The differential pattern of expression in each cancer—or gene expression signature—may then be used to generate a risk score that is predictive of cancer survival. The level of expression of a biomarker may be increased or decreased in a subject relative to other subjects with cancer. The expression of a biomarker may be higher in long-term survivors than in short-term survivors. Alternatively, the expression of a biomarker may be higher in short-term survivors than in long-term survivors.

The differential expression of a plurality of biomarkers may be measured by a variety of techniques that are well known in the art. Quantifying the levels of the messenger RNA (mRNA) of a biomarker may be used to measure the expression of the biomarker. Alternatively, quantifying the levels of the protein product of a biomarker may be to measure the expression of the biomarker. Additional information regarding the methods discussed below may be found in Ausubel et al., (2003) Current Protocols in Molecular Biology, John Wiley & Sons, New York, N.Y., or Sambrook et al. (1989) Molecular Cloning: A Laboratory Manual, Cold Spring Harbor Press, Cold Spring Harbor, N.Y. One skilled in the art will know which parameters may be manipulated to optimize detection of the mRNA or protein of interest.

A nucleic acid microarray may be used to quantify the differential expression of a plurality of biomarkers. Microarray analysis may be performed using commercially available equipment, following manufacturer's protocols, such as by using the Affymetrix GeneChip® technology (Santa Clara, Calif.) or the Microarray System from Incyte (Fremont, Calif.). Typically, single-stranded nucleic acids (e.g., cDNAs or oligonucleotides) are plated, or arrayed, on a microchip substrate. The arrayed sequences are then hybridized with specific nucleic acid probes from the cells of interest. Fluorescently labeled cDNA probes may be generated through incorporation of fluorescently labeled deoxynucleotides by reverse transcription of RNA extracted from the cells of interest. Alternatively, the RNA may be amplified by in vitro transcription and labeled with a marker, such as biotin. The labeled probes are then hybridized to the immobilized nucleic acids on the microchip under highly stringent conditions. After stringent washing to remove the non-specifically bound probes, the chip is scanned by confocal laser microscopy or by another detection method, such as a CCD camera. The raw fluorescence intensity data in the hybridization files are generally preprocessed with the robust multichip average (RMA) algorithm to generate expression values.

Quantitative real-time PCR (QRT-PCR) may also be used to measure the differential expression of a plurality of biomarkers. In QRT-PCR, the RNA template is generally reverse transcribed into cDNA, which is then amplified via a PCR reaction. The amount of PCR product is followed cycle-by-cycle in real time, which allows for determination of the initial concentrations of mRNA. To measure the amount of PCR product, the reaction may be performed in the presence of a fluorescent dye, such as SYBR Green, which binds to double-stranded DNA. The reaction may also be performed with a fluorescent reporter probe that is specific for the DNA being amplified. A non-limiting example of a fluorescent reporter probe is a TagMan® probe (Applied Biosystems, Foster City, Calif.). The fluorescent reporter probe fluoresces when the quencher is removed during the PCR extension cycle. Muliplex QRT-PCR may be performed by using multiple gene-specific reporter probes, each of which contains a different fluorophore. Fluorescence values are recorded during each cycle and represent the amount of product amplified to that point in the amplification reaction. To minimize errors and reduce any sample-to-sample variation, QRT-PCR is typically performed using a reference standard. The ideal reference standard is expressed at a constant level among different tissues, and is unaffected by the experimental treatment. Suitable reference standards include, but are not limited to, mRNAs for the housekeeping genes glyceraldehyde-3-phosphate-dehydrogenase (GAPDH) and beta-actin. The level of mRNA in the original sample or the fold change in expression of each biomarker may be determined using calculations well known in the art.

Immunohistochemical staining may also be used to measure the differential expression of a plurality of biomarkers. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

An enzyme-linked immunosorbent assay, or ELISA, may be used to measure the differential expression of a plurality of biomarkers. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

An antibody microarray may also be used to measure the differential expression of a plurality of biomarkers. For this, a plurality of antibodies is arrayed and covalently attached to the surface of the microarray or biochip. A protein extract containing the biomarker proteins of interest is generally labeled with a fluorescent dye. The labeled biomarker proteins are incubated with the antibody microarray. After washes to remove the unbound proteins, the microarray is scanned. The raw fluorescent intensity data maybe converted into expression values using means known in the art.

Luminex multiplexing microspheres may also be used to measure the differential expression of a plurality of biomarkers. These microscopic polystyrene beads are internally color-coded with fluorescent dyes, such that each bead has a unique spectral signature (of which there are up to 100). Beads with the same signature are tagged with a specific oligonucleotide or specific antibody that will bind the target of interest (i.e., biomarker mRNA or protein, respectively). The target, in turn, is also tagged with a fluorescent reporter. Hence, there are two sources of color, one from the bead and the other from the reporter molecule on the target. The beads are then incubated with the sample containing the targets, of which up 100 may be detected in one well. The small size/surface area of the beads and the three dimensional exposure of the beads to the targets allows for nearly solution-phase kinetics during the binding reaction. The captured targets are detected by high-tech fluidics based upon flow cytometry in which lasers excite the internal dyes that identify each bead and also any reporter dye captured during the assay. The data from the acquisition files may be converted into expression values using means known in the art.

In situ hybridization may also be used to measure the differential expression of a plurality of biomarkers. This method permits the localization of mRNAs of interest in the cells of a tissue section. For this method, the tissue may be frozen, or fixed and embedded, and then cut into thin sections, which are arrayed and affixed on a solid surface. The tissue sections are incubated with a labeled antisense probe that will hybridize with an mRNA of interest. The hybridization and washing steps are generally performed under highly stringent conditions. The probe may be labeled with a fluorophore or a small tag (such as biotin or digoxigenin) that may be detected by another protein or antibody, such that the labeled hybrid may be detected and visualized under a microscope. Multiple mRNAs may be detected simultaneously, provided each antisense probe has a distinguishable label. The hybridized tissue array is generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for each biomarker.

The number of biomarkers whose expression is measured in a sample of cells from a subject with cancer may vary. Since the predicted score of survival is based upon the differential expression of the biomarkers, a higher degree of accuracy should be attained when the expression of more biomarkers is measured.

Obtaining a Sample of Cells from a Subject with Cancer

The expression of a plurality of biomarkers will be measured in a sample of cells from a subject with cancer. The type and classification of the cancer can and will vary. The cancer may be an early stage cancer, i.e., stage I or stage II, or it may be a late stage cancer, i.e., stage Ill or stage IV.

Generally, the sample of cells or tissue sample will be obtained from the subject with cancer by biopsy or surgical resection. The type of biopsy can and will vary, depending upon the location and nature of the cancer. A sample of cells, tissue, or fluid may be removed by needle aspiration biopsy. For this, a fine needle attached to a syringe is inserted through the skin and into the organ or tissue of interest. The needle is typically guided to the region of interest using ultrasound or computed tomography (CT) imaging. Once the needle is inserted into the tissue, a vacuum is created with the syringe such that cells or fluid may be sucked through the needle and collected in the syringe. A sample of cells or tissue may also be removed by incisional or core biopsy. For this, a cone, a cylinder, or a tiny bit of tissue is removed from the region of interest. CT imaging, ultrasound, or an endoscope is generally used to guide this type of biopsy. Lastly, the entire cancerous lesion may be removed by excisional biopsy or surgical resection.

Once a sample of cells or sample of tissue is removed from the subject with cancer, it may be processed for the isolation of RNA or protein using techniques well known in the art and disclosed in standard molecular biology reference books, such as Ausubel et al., (2003) Current Protocols in Molecular Biology, John Wiley & Sons, New York, N.Y. A sample of tissue may also be stored or flash frozen and stored at −80° C. for later use. The biopsied tissue sample may also be fixed with a fixative, such as formaldehyde, paraformaldehyde, or acetic acid/ethanol. The fixed tissue sample may be embedded in wax (paraffin) or a plastic resin. The embedded tissue sample (or frozen tissue sample) may be cut into thin sections. RNA or protein may also be extracted from a fixed or wax-embedded tissue sample.

The subject with cancer will generally be a mammalian subject. Mammals may include primates, livestock animals, and companion animals. Non-limiting examples include: Primates may include humans, apes, monkeys, and gibbons; Livestock animals may include horses, cows, goats, sheep, deer and pigs; Companion animals may include dogs, cats, rabbits, and rodents (including mice, rats, and guinea pigs). In an exemplary embodiment, the subject is a human.

Generating a Risk Score

The biomarkers of this invention are related to cancer survival. The differential patterns of expression of a plurality of these biomarkers may be used to predict the survival outcome of a subject with cancer. Certain biomarkers tend to be over-expressed in long-term survivors, whereas other biomarkers tend to be over-expressed in short-term survivors. The unique pattern of expression of a plurality of biomarkers in a subject (i.e., the expression signature) may be used to generate a risk score of survival. Subjects with a high risk score may have a short survival time (<2 years) after surgical resection. Subjects with a low risk score may have a longer survival time (>5 years) after resection.

Regardless of the technique used to measure the differential expression of a plurality of biomarkers, the expression of each biomarker typically will be converted into an expression value. These expression values then will be used to calculate a risk score of survival for a subject with cancer using statistical methods well known in the art. The risk scores may be calculated using a principal components analysis. The risk scores may also be calculated using a univariate Cox regression analysis. In one preferred embodiment, the risk scores may be calculated using a partial Cox regression analysis.

The scores generated by a partial Cox regression analysis fall into two groups: 1) those having a positive value; and 2) those having a negative value. A risk score having a positive value is associated with a short survival time, and a risk score having a negative value is associated with a long survival time.

In one embodiment of this method, a tissue sample may be removed by surgical resection from a subject with an early stage cancer. The sample of tissue may be stored in RNAlater or flash frozen, such that RNA may be isolated at a later date. The RNA may be used as template for QRT-PCR in which the expression of a plurality of biomarkers is analyzed, and the expression data are used to derive a risk score using the partial Cox regression classification method. The risk score may be used to predict whether the subject will be a short-term or a long-term cancer survivor.

In an especially preferred embodiment of this method, a sample of tissue may be collected from a subject with an early stage cancer. RNA may be isolated from the tissue and used to generate labeled probes for a nucleic acid microarray analysis. The expression values generated from the microarray analysis may be used to derive a risk score using the partial Cox regression classification method. The risk score may be used to predict whether the subject will be a short-term or a long-term cancer survivor.

Method for Determining the Prognosis of a Subject with Cancer

Another aspect of the invention provides a method for determining the prognosis of a subject with a cancer. The method comprises measuring the differential expression of one or more biomarkers in a sample of cells from the subject. The differential expression of each biomarker is converted into an expression value, and the expression values are used to derive a score for that subject using a statistical method, as detailed above. A score having a positive value is indicative of a poor prognosis or a poor outcome, whereas a score having a negative value is indicative of a good prognosis or a good outcome.

In one embodiment of this method, an expression signature for a subject with an early stage cancer is generated by nucleic acid microarray analysis, and the expression values are used to calculate a score. The calculated score may be used to predict whether the subject will have a good prognosis or a poor prognosis of cancer outcome.

Method for Selecting a Treatment for a Subject with Cancer

A further aspect of the invention provides a method for selecting an effective treatment for a subject with cancer. Once a risk score has been calculated for a subject, that information may be used to decide upon an appropriate course of treatment for the subject. A subject having a positive risk score (i.e., short survival time or poor prognosis) may benefit from an aggressive therapeutic regime. An aggressive therapeutic regime may comprise the appropriate chemotherapy agent or agents. An aggressive therapeutic regime may also comprise radiation therapy. The treatment regime can and will vary, depending upon the type and stage of cancer. A subject having a negative risk score (i.e., long survival time or good prognosis) may not need additional treatment, since the subject is not likely to develop a recurrent cancer.

The cells are maintained under conditions in which the one or more agents inhibits expression or activity of the microRNAs, inhibits expression of one or more target genes of the microRNAs, or inhibits a combination thereof, thereby inhibiting proliferation of the cell.

Methods of identifying an agent that can be used to inhibit proliferation of a cancer cell are also provided. The method comprises contacting one or more microRNAs with an agent to be assessed; contacting one or more target genes of one or more with an agent to be assessed; or contacting a combination thereof. If expression of the microRNAs is inhibited in the presence of the agent; of if expression of the target genes is enhanced in the presence of the agent, or a combination thereof occurs in the presence of the agent, then the agent can be used to inhibit proliferation of a follicular thyroid carcinoma cell.

Method of Identifying Therapeutic Agents

Also provided herein are methods of identifying an agent that can be used to treat CRC. The method comprises contacting one or more microRNAs with an agent to be assessed; contacting one or more target genes of one or more microRNAs; or contacting a combination thereof. If expression of the microRNAs is inhibited in the presence of the agent; of if expression of the target genes is enhanced in the presence of the agent, or a combination thereof occurs in the presence of the agent, then the agent can be used to inhibit proliferation of a follicular thyroid carcinoma cell.

Agents that can be assessed in the methods provided herein include miRNA inhibitors. Other examples of such agents include pharmaceutical agents, drugs, chemical compounds, ionic compounds, organic compounds, organic ligands, including cofactors, saccharides, recombinant and synthetic peptides, proteins, peptoids, nucleic acid sequences, including genes, nucleic acid products, and antibodies and antigen binding fragments thereof. Such agents can be individually screened or one or more compound(s) can be tested simultaneously in accordance with the methods herein. Large combinatorial libraries of compounds (e.g., organic compounds, recombinant or synthetic peptides, peptoids, nucleic acids) produced by combinatorial chemical synthesis or other methods can be tested. Where compounds selected from a combinatorial library carry unique tags, identification of individual compounds by chromatographic methods is possible. Chemical libraries, microbial broths and phage display libraries can also be tested (screened) in accordance with the methods herein.

Kit for Predicting Survival or Prognosis of a Subject with Cancer

A further aspect of the invention provides kits for predicting survival or prognosis of a subject with cancer. A kit comprises a plurality of agents for measuring the differential expression of one or more biomarkers, means for converting the expression data into expression values, and means for analyzing the expression values to generate scores that predict survival or prognosis. The agents in the kit for measuring biomarker expression may comprise an array of polynucleotides complementary to the mRNAs of the biomarkers. In another embodiment, the agents in the kit for measuring biomarker expression may comprise a plurality of PCR probes and/or primers for QRT-PCR.

The invention is also directed to kits for detecting CRC an individual comprising one or more reagents for detecting 1) one or more microRNAs; 2) one or more target genes of one or more microRNAs; 3) one or more polypeptides expressed by the target genes or 4) a combination thereof. For example, the kit can comprise hybridization probes, restriction enzymes (e.g., for RFLP analysis), allele-specific oligonucleotides, and antibodies that bind to the polypeptide expressed by the target gene.

In a particular embodiment, the kit comprises at least contiguous nucleotide sequence that is substantially or completely complementary to a region one or more of the microRNAs. In one embodiment, one or reagents in the kit are labeled, and thus, the kits can further comprise agents capable of detecting the label. The kit can further comprise instructions for detecting CRC using the components of the kit.

Nucleic Acid Array

Another aspect of the invention provides for a nucleic acid array comprising polynucleotides that hybridize to the mRNAs of biomarkers of the invention. Generally speaking, the nucleic acid array is comprised of a substrate having at least one address. Nucleic acid arrays are commonly known in the art, and moreover, substrates that comprise nucleic acid arrays are also well known in the art. Non-limiting examples of substrate materials include glass and plastic. A substrate may be shaped like a slide or a chip (i.e. a quadrilateral shape), or alternatively, a substrate may be shaped like a well.

The array of the present invention is comprised of at least one address, wherein the address has disposed thereon a nucleic acid that can hybridize to the mRNA of a biomarker of the invention. In one embodiment, the array is comprised of multiple addresses, wherein each address has disposed thereon a nucleic acid that can hybridize to the mRNA of a biomarker for predicting survival of a subject with a lung cancer. The array may also comprise one or more addresses wherein the address has disposed thereon a control nucleic acid. The control may be an internal control (i.e. a control for the array itself) and/or an external control (i.e. a control for the sample applied to the array). An array typically is comprised from between about 1 to about 10,000 addresses. In one embodiment, the array is comprised from between about 10 to about 8,000 addresses. In another embodiment, the array is comprised of no more than 500 addresses. In an alternative embodiment, the array is comprised of no less than 500 addresses. Methods of using nucleic acid arrays are well known in the art.

Methods of Use

In one aspect, there is provided herein a method of diagnosing whether a subject has, or is at risk for developing MM and/or MGUS, comprising measuring the level of at least one gene product in a test sample from the subject and comparing the level of the gene product in the test sample to the level of a corresponding gene product in a control sample. As used herein, a “subject” can be any mammal that has, or is suspected of having, MM and/or MGUS. In a particular embodiment, the subject is a human who has, or is suspected of having, MM and/or MGUS.

The level of at least one gene product can be measured in cells of a biological sample obtained from the subject. For example, a tissue sample can be removed from a subject suspected of having MM and/or MGUS associated with by conventional sampling techniques. In another example, a blood sample can be removed from the subject, and white blood cells can be isolated for DNA extraction by standard techniques. The blood or tissue sample is preferably obtained from the subject prior to initiation of radiotherapy, chemotherapy or other therapeutic treatment. A corresponding control tissue or blood sample can be obtained from unaffected tissues of the subject, from a normal human individual or population of normal individuals, or from cultured cells corresponding to the majority of cells in the subject's sample. The control tissue or blood sample is then processed along with the sample from the subject, so that the levels of gene product produced from a given gene in cells from the subject's sample can be compared to the corresponding gene product levels from cells of the control sample.

An alteration (i.e., an increase or decrease) in the level of a gene product in the sample obtained from the subject, relative to the level of a corresponding gene product in a control sample, is indicative of the presence of MM and/or MGUS in the subject. In one embodiment, the level of the at least one gene product in the test sample is greater than the level of the corresponding gene product in the control sample (i.e., expression of the gene product is “up-regulated”). As used herein, expression of a gene product is “up-regulated” when the amount of gene product in a cell or tissue sample from a subject is greater than the amount the same gene product in a control cell or tissue sample. In another embodiment, the level of the at least one gene product in the test sample is less than the level of the corresponding gene product in the control sample (i.e., expression of the gene product is “down-regulated”). As used herein, expression of a gene is “down-regulated” when the amount of gene product produced from that gene in a cell or tissue sample from a subject is less than the amount produced from the same gene in a control cell or tissue sample. The relative gene expression in the control and normal samples can be determined with respect to one or more RNA expression standards. The standards can comprise, for example, a zero gene expression level, the gene expression level in a standard cell line, or the average level of gene expression previously obtained for a population of normal human controls.

The level of a gene product in a sample can be measured using any technique that is suitable for detecting RNA expression levels in a biological sample. Suitable techniques for determining RNA expression levels in cells from a biological sample (e.g., Northern blot analysis, RT-PCR, in situ hybridization) are well known to those of skill in the art. In a particular embodiment, the level of at least one gene product is detected using Northern blot analysis. For example, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters. The RNA is then immobilized on the filters by heating. Detection and quantification of specific RNA is accomplished using appropriately labeled DNA or RNA probes complementary to the RNA in question. See, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

Suitable probes for Northern blot hybridization of a given gene product can be produced from the nucleic acid sequences of the given gene product. Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11, the disclosures of which are incorporated herein by reference.

For example, the nucleic acid probe can be labeled with, e.g., a radionuclide, such as ³H, ³²P, ³³P, ¹⁴C, or ³⁵S; a heavy metal; or a ligand capable of functioning as a specific binding pair member for a labeled ligand (e.g., biotin, avidin or an antibody), a fluorescent molecule, a chemiluminescent molecule, an enzyme or the like.

Probes can be labeled to high specific activity by either the nick translation method of Rigby et al. (1977), J. Mol. Biol. 113:237-251 or by the random priming method of Fienberg et al. (1983), Anal. Biochem. 132:6-13, the entire disclosures of which are incorporated herein by reference. The latter is the method of choice for synthesizing ³²P-labeled probes of high specific activity from single-stranded DNA or from RNA templates. For example, by replacing preexisting nucleotides with highly radioactive nucleotides according to the nick translation method, it is possible to prepare ³²P-labeled nucleic acid probes with a specific activity well in excess of 10⁸ cpm/microgram. Autoradiographic detection of hybridization can then be performed by exposing hybridized filters to photographic film. Densitometric scanning of the photographic films exposed by the hybridized filters provides an accurate measurement of gene transcript levels. Using another approach, gene transcript levels can be quantified by computerized imaging systems, such the Molecular Dynamics 400-B 2D Phosphorimager available from Amersham Biosciences, Piscataway, N.J.

Where radionuclide labeling of DNA or RNA probes is not practical, the random-primer method can be used to incorporate an analogue, for example, the dTTP analogue 5-(N-(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g., anti-biotin antibodies) coupled to fluorescent dyes or enzymes that produce color reactions.

In addition to Northern and other RNA hybridization techniques, determining the levels of RNA transcripts can be accomplished using the technique of in situ hybridization. This technique requires fewer cells than the Northern blotting technique, and involves depositing whole cells onto a microscope cover slip and probing the nucleic acid content of the cell with a solution containing radioactive or otherwise labeled nucleic acid (e.g., cDNA or RNA) probes. This technique is particularly well-suited for analyzing tissue biopsy samples from subjects. The practice of the in situ hybridization technique is described in more detail in U.S. Pat. No. 5,427,916, the entire disclosure of which is incorporated herein by reference. Suitable probes for in situ hybridization of a given gene product can be produced from the nucleic acid sequences.

The relative number of gene transcripts in cells can also be determined by reverse transcription of gene transcripts, followed by amplification of the reverse-transcribed transcripts by polymerase chain reaction (RT-PCR). The levels of gene transcripts can be quantified in comparison with an internal standard, for example, the level of mRNA from a “housekeeping” gene present in the same sample. A suitable “housekeeping” gene for use as an internal standard includes, e.g., myosin or glyceraldehyde-3-phosphate dehydrogenase (G3PDH). The methods for quantitative RT-PCR and variations thereof are within the skill in the art.

In some instances, it may be desirable to simultaneously determine the expression level of a plurality of different gene products in a sample. In other instances, it may be desirable to determine the expression level of the transcripts of all known genes correlated with a cancer. Assessing cancer-specific expression levels for hundreds of genes is time consuming and requires a large amount of total RNA (at least 20 μg for each Northern blot) and autoradiographic techniques that require radioactive isotopes.

To overcome these limitations, an oligolibrary, in microchip format (i.e., a microarray), may be constructed containing a set of probe oligodeoxynucleotides that are specific for a set of genes or gene products. Using such a microarray, the expression level of multiple microRNAs in a biological sample can be determined by reverse transcribing the RNAs to generate a set of target oligodeoxynucleotides, and hybridizing them to probe oligodeoxynucleotides on the microarray to generate a hybridization, or expression, profile. The hybridization profile of the test sample can then be compared to that of a control sample to determine which microRNAs have an altered expression level. As used herein, “probe oligonucleotide” or “probe oligodeoxynucleotide” refers to an oligonucleotide that is capable of hybridizing to a target oligonucleotide. “Target oligonucleotide” or “target oligodeoxynucleotide” refers to a molecule to be detected (e.g., via hybridization). By “specific probe oligonucleotide” or “probe oligonucleotide specific for a gene product” is meant a probe oligonucleotide that has a sequence selected to hybridize to a specific gene product, or to a reverse transcript of the specific gene product.

An “expression profile” or “hybridization profile” of a particular sample is essentially a fingerprint of the state of the sample; while two states may have any particular gene similarly expressed, the evaluation of a number of genes simultaneously allows the generation of a gene expression profile that is unique to the state of the cell. That is, normal cells may be distinguished from MM and/or MGUS cells, and within MM and/or MGUS cells, different prognosis states (good or poor long term survival prospects, for example) may be determined. By comparing expression profiles of MM and/or MGUS cells in different states, information regarding which genes are important (including both up- and down-regulation of genes) in each of these states is obtained. The identification of sequences that are differentially expressed in MM and/or MGUS cells or normal cells, as well as differential expression resulting in different prognostic outcomes, allows the use of this information in a number of ways. For example, a particular treatment regime may be evaluated (e.g., to determine whether a chemotherapeutic drug act to improve the long-term prognosis in a particular patient). Similarly, diagnosis may be done or confirmed by comparing patient samples with the known expression profiles. Furthermore, these gene expression profiles (or individual genes) allow screening of drug candidates that suppress the MM and/or MGUS expression profile or convert a poor prognosis profile to a better prognosis profile.

Accordingly, the invention provides methods of diagnosing whether a subject has, or is at risk for developing, MM and/or MGUS, comprising reverse transcribing RNA from a test sample obtained from the subject to provide a set of target oligo-deoxynucleotides, hybridizing the target oligo-deoxynucleotides to a microarray comprising miRNA-specific probe oligonucleotides to provide a hybridization profile for the test sample, and comparing the test sample hybridization profile to a hybridization profile generated from a control sample, wherein an alteration in the signal of at least one miRNA is indicative of the subject either having, or being at risk for developing, MM and/or MGUS.

The invention also provides methods of diagnosing a MM and/or MGUS associated with one or more prognostic markers, comprising measuring the level of at least one gene product in a MM and/or MGUS test sample from a subject and comparing the level of the at least one gene product in the MM and/or MGUS test sample to the level of a corresponding gene product in a control sample. An alteration (e.g., an increase, a decrease) in the signal of at least one gene product in the test sample relative to the control sample is indicative of the subject either having, or being at risk for developing, MM and/or MGUS associated with the one or more prognostic markers.

The MM and/or MGUS can be associated with one or more prognostic markers or features, including, a marker associated with an adverse (i.e., negative) prognosis, or a marker associated with a good (i.e., positive) prognosis. In certain embodiments, the MM and/or MGUS that is diagnosed using the methods described herein are associated with one or more adverse prognostic features.

Particular microRNAs whose expression is altered in MM and/or MGUS cells associated with each of these prognostic markers are described herein. In one embodiment, the level of the at least one gene product is measured by reverse transcribing RNA from a test sample obtained from the subject to provide a set of target oligodeoxynucleotides, hybridizing the target oligodeoxynucleotides to a microarray that comprises miRNA-specific probe oligonucleotides to provide a hybridization profile for the test sample, and comparing the test sample hybridization profile to a hybridization profile generated from a control sample.

Without wishing to be bound by any one theory, it is believed that alterations in the level of one or more gene products in cells can result in the deregulation of one or more intended targets for these gene products, which can lead to the formation of MM and/or MGUS. Therefore, altering the level of the gene product (e.g., by decreasing the level of a gene product that is up-regulated in MM and/or MGUS cells, by increasing the level of a gene product that is down-regulated in cancer cells) may successfully treat the MM and/or MGUS. Examples of putative gene targets for gene products that are deregulated in MM and/or MGUS cells are described herein.

Accordingly, the present invention encompasses methods of treating MM and/or MGUS in a subject, wherein at least one gene product is de-regulated (e.g., down-regulated, up-regulated) in the cancer cells of the subject. When the at least one isolated gene product is down-regulated in the MM and/or MGUS cells, the method comprises administering an effective amount of the at least one isolated gene product such that proliferation of cancer cells in the subject is inhibited. When the at least one isolated gene product is up-regulated in the cancer cells, the method comprises administering to the subject an effective amount of at least one compound for inhibiting expression of the at least one gene, referred to herein as gene expression inhibition compounds, such that proliferation of MM and/or MGUS cells is inhibited.

The terms “treat”, “treating” and “treatment”, as used herein, refer to ameliorating symptoms associated with a disease or condition, for example, MM and/or MGUS, including preventing or delaying the onset of the disease symptoms, and/or lessening the severity or frequency of symptoms of the disease or condition. The terms “subject” and “individual” are defined herein to include animals, such as mammals, including but not limited to, primates, cows, sheep, goats, horses, dogs, cats, rabbits, guinea pigs, rats, mice or other bovine, ovine, equine, canine, feline, rodent, or murine species. In a preferred embodiment, the animal is a human.

As used herein, an “effective amount” of an isolated gene product is an amount sufficient to inhibit proliferation of a cancer cell in a subject suffering from MM and/or MGUS L. One skilled in the art can readily determine an effective amount of an gene product to be administered to a given subject, by taking into account factors, such as the size and weight of the subject; the extent of disease penetration; the age, health and sex of the subject; the route of administration; and whether the administration is regional or systemic.

For example, an effective amount of an isolated gene product can be based on the approximate or estimated body weight of a subject to be treated. Preferably, such effective amounts are administered parenterally or enterally, as described herein. For example, an effective amount of the isolated gene product is administered to a subject can range from about 5-3000 micrograms/kg of body weight, from about 700-1000 micrograms/kg of body weight, or greater than about 1000 micrograms/kg of body weight.

One skilled in the art can also readily determine an appropriate dosage regimen for the administration of an isolated gene product to a given subject. For example, a gene product can be administered to the subject once (e.g., as a single injection or deposition). Alternatively, a gene product can be administered once or twice daily to a subject for a period of from about three to about twenty-eight days, more particularly from about seven to about ten days. In a particular dosage regimen, a gene product is administered once a day for seven days. Where a dosage regimen comprises multiple administrations, it is understood that the effective amount of the gene product administered to the subject can comprise the total amount of gene product administered over the entire dosage regimen.

As used herein, an “isolated” gene product is one which is synthesized, or altered or removed from the natural state through human intervention. For example, a synthetic gene product, or a gene product partially or completely separated from the coexisting materials of its natural state, is considered to be “isolated.” An isolated gene product can exist in substantially-purified form, or can exist in a cell into which the gene product has been delivered. Thus, a gene product which is deliberately delivered to, or expressed in, a cell is considered an “isolated” gene product. A gene product produced inside a cell from a precursor molecule is also considered to be “isolated” molecule.

Isolated gene products can be obtained using a number of standard techniques. For example, the gene products can be chemically synthesized or recombinantly produced using methods known in the art. In one embodiment, gene products are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), ChemGenes (Ashland, Mass., U.S.A.) and Cruachem (Glasgow, UK).

Alternatively, the gene products can be expressed from recombinant circular or linear DNA plasmids using any suitable promoter. Suitable promoters for expressing RNA from a plasmid include, e.g., the U6 or H1 RNA pol III promoter sequences, or the cytomegalovirus promoters. Selection of other suitable promoters is within the skill in the art. The recombinant plasmids of the invention can also comprise inducible or regulatable promoters for expression of the gene products in cancer cells.

The gene products that are expressed from recombinant plasmids can be isolated from cultured cell expression systems by standard techniques. The gene products which are expressed from recombinant plasmids can also be delivered to, and expressed directly in, the cancer cells. The use of recombinant plasmids to deliver the gene products to cancer cells is discussed in more detail below.

The gene products can be expressed from a separate recombinant plasmid, or they can be expressed from the same recombinant plasmid. In one embodiment, the gene products are expressed as RNA precursor molecules from a single plasmid, and the precursor molecules are processed into the functional gene product by a suitable processing system, including, but not limited to, processing systems extant within a cancer cell. Other suitable processing systems include, e.g., the in vitro Drosophila cell lysate system (e.g., as described in U.S. Published Patent Application No. 2002/0086356 to Tuschl et al., the entire disclosure of which are incorporated herein by reference) and the E. coli RNAse III system (e.g., as described in U.S. Published Patent Application No. 2004/0014113 to Yang et al., the entire disclosure of which are incorporated herein by reference).

Selection of plasmids suitable for expressing the gene products, methods for inserting nucleic acid sequences into the plasmid to express the gene products, and methods of delivering the recombinant plasmid to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee et al. (2002), Nat. Biotechnol. 20:500-505; and Paul et al. (2002), Nat. Biotechnol. 20:505-508, the entire disclosures of which are incorporated herein by reference.

In one embodiment, a plasmid expressing the gene products comprises a sequence encoding a precursor RNA under the control of the CMV intermediate-early promoter. As used herein, “under the control” of a promoter means that the nucleic acid sequences encoding the gene product are located 3′ of the promoter, so that the promoter can initiate transcription of the gene product coding sequences.

The gene products can also be expressed from recombinant viral vectors. It is contemplated that the gene products can be expressed from two separate recombinant viral vectors, or from the same viral vector. The RNA expressed from the recombinant viral vectors can either be isolated from cultured cell expression systems by standard techniques, or can be expressed directly in cancer cells. The use of recombinant viral vectors to deliver the gene products to cancer cells is discussed in more detail below.

The recombinant viral vectors of the invention comprise sequences encoding the gene products and any suitable promoter for expressing the RNA sequences. Suitable promoters include, for example, the U6 or H1 RNA pol III promoter sequences, or the cytomegalovirus promoters. Selection of other suitable promoters is within the skill in the art. The recombinant viral vectors of the invention can also comprise inducible or regulatable promoters for expression of the gene products in a cancer cell.

Any viral vector capable of accepting the coding sequences for the gene products can be used; for example, vectors derived from adenovirus (AV); adeno-associated virus (AAV); retroviruses (e.g., lentiviruses (LV), Rhabdoviruses, murine leukemia virus); herpes virus, and the like. The tropism of the viral vectors can be modified by pseudotyping the vectors with envelope proteins or other surface antigens from other viruses, or by substituting different viral capsid proteins, as appropriate.

For example, lentiviral vectors of the invention can be pseudotyped with surface proteins from vesicular stomatitis virus (VSV), rabies, Ebola, Mokola, and the like. AAV vectors of the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors that express different capsid protein serotypes are within the skill in the art; see, e.g., Rabinowitz, J. E., et al. (2002), J. Virol. 76:791-801, the entire disclosure of which is incorporated herein by reference.

Selection of recombinant viral vectors suitable for use in the invention, methods for inserting nucleic acid sequences for expressing RNA into the vector, methods of delivering the viral vector to the cells of interest, and recovery of the expressed RNA products are within the skill in the art. See, for example, Dornburg (1995), Gene Therap. 2:301-310; Eglitis (1988), Biotechniques 6:608-614; Miller (1990), Hum. Gene Therap. 1:5-14; and Anderson (1998), Nature 392:25-30, the entire disclosures of which are incorporated herein by reference.

Om certain embodiments, suitable viral vectors are those derived from AV and AAV. A suitable AV vector for expressing the gene products, a method for constructing the recombinant AV vector, and a method for delivering the vector into target cells, are described in Xia et al. (2002), Nat. Biotech. 20:1006-1010, the entire disclosure of which is incorporated herein by reference. Suitable AAV vectors for expressing the gene products, methods for constructing the recombinant AAV vector, and methods for delivering the vectors into target cells are described in Samulski et al. (1987), J. Virol. 61:3096-3101; Fisher et al. (1996), J. Virol., 70:520-532; Samulski et al. (1989), J. Virol. 63:3822-3826; U.S. Pat. No. 5,252,479; U.S. Pat. No. 5,139,941; International Patent Application No. WO 94/13788; and International Patent Application No. WO 93/24641, the entire disclosures of which are incorporated herein by reference.

In a certain embodiment, a recombinant AAV viral vector of the invention comprises a nucleic acid sequence encoding a precursor RNA in operable connection with a polyT termination sequence under the control of a human U6 RNA promoter. As used herein, “in operable connection with a polyT termination sequence” means that the nucleic acid sequences encoding the sense or antisense strands are immediately adjacent to the polyT termination signal in the 5′ direction. During transcription of the sequences from the vector, the polyT termination signals act to terminate transcription.

In other embodiments of the treatment methods of the invention, an effective amount of at least one compound which inhibits expression can also be administered to the subject. As used herein, “inhibiting gene expression” means that the production of the active, mature form of gene product after treatment is less than the amount produced prior to treatment. One skilled in the art can readily determine whether expression has been inhibited in a cancer cell, using for example the techniques for determining transcript level discussed above for the diagnostic method. Inhibition can occur at the level of gene expression (i.e., by inhibiting transcription of a gene encoding the gene product) or at the level of processing (e.g., by inhibiting processing of a precursor into a mature, active gene product).

As used herein, an “effective amount” of a compound that inhibits expression is an amount sufficient to inhibit proliferation of a cancer cell in a subject suffering from a cancer associated with a cancer-associated chromosomal feature. One skilled in the art can readily determine an effective amount of an expression-inhibiting compound to be administered to a given subject, by taking into account factors, such as the size and weight of the subject; the extent of disease penetration; the age, health and sex of the subject; the route of administration; and whether the administration is regional or systemic.

For example, an effective amount of the expression-inhibiting compound can be based on the approximate or estimated body weight of a subject to be treated. Such effective amounts are administered parenterally or enterally, among others, as described herein. For example, an effective amount of the expression-inhibiting compound administered to a subject can range from about 5-3000 micrograms/kg of body weight, from about 700-1000 micrograms/kg of body weight, or it can be greater than about 1000 micrograms/kg of body weight.

One skilled in the art can also readily determine an appropriate dosage regimen for administering a compound that inhibits expression to a given subject. For example, an expression-inhibiting compound can be administered to the subject once (e.g., as a single injection or deposition). Alternatively, an expression-inhibiting compound can be administered once or twice daily to a subject for a period of from about three to about twenty-eight days, more preferably from about seven to about ten days. In a particular dosage regimen, an expression-inhibiting compound is administered once a day for seven days. Where a dosage regimen comprises multiple administrations, it is understood that the effective amount of the expression-inhibiting compound administered to the subject can comprise the total amount of compound administered over the entire dosage regimen.

Suitable compounds for inhibiting expression include double-stranded RNA (such as short- or small-interfering RNA or “siRNA”), antisense nucleic acids, and enzymatic RNA molecules, such as ribozymes. Each of these compounds can be targeted to a given gene product and destroy or induce the destruction of the target gene product.

For example, expression of a given gene can be inhibited by inducing RNA interference of the gene with an isolated double-stranded RNA (“dsRNA”) molecule which has at least 90%, for example at least 95%, at least 98%, at least 99% or 100%, sequence homology with at least a portion of the gene product. In a particular embodiment, the dsRNA molecule is a “short or small interfering RNA” or “siRNA.”

siRNA useful in the present methods comprise short double-stranded RNA from about 17 nucleotides to about 29 nucleotides in length, preferably from about 19 to about 25 nucleotides in length. The siRNA comprise a sense RNA strand and a complementary antisense RNA strand annealed together by standard Watson-Crick base-pairing interactions (hereinafter “base-paired”). The sense strand comprises a nucleic acid sequence which is substantially identical to a nucleic acid sequence contained within the target gene product.

As used herein, a nucleic acid sequence in an siRNA which is “substantially identical” to a target sequence contained within the target mRNA is a nucleic acid sequence that is identical to the target sequence, or that differs from the target sequence by one or two nucleotides. The sense and antisense strands of the siRNA can comprise two complementary, single-stranded RNA molecules, or can comprise a single molecule in which two complementary portions are base-paired and are covalently linked by a single-stranded “hairpin” area.

The siRNA can also be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides.

One or both strands of the siRNA can also comprise a 3′ overhang. As used herein, a “3′ overhang” refers to at least one unpaired nucleotide extending from the 3′-end of a duplexed RNA strand. Thus, in certain embodiments, the siRNA comprises at least one 3′ overhang of from 1 to about 6 nucleotides (which includes ribonucleotides or deoxyribonucleotides) in length, from 1 to about 5 nucleotides in length, from 1 to about 4 nucleotides in length, or from about 2 to about 4 nucleotides in length. In a particular embodiment, the 3′ overhang is present on both strands of the siRNA, and is 2 nucleotides in length. For example, each strand of the siRNA can comprise 3′ overhangs of dithymidylic acid (“TT”) or diuridylic acid (“uu”).

The siRNA can be produced chemically or biologically, or can be expressed from a recombinant plasmid or viral vector, as described above for the isolated

Exemplary methods for producing and testing dsRNA or siRNA molecules are described in U.S. Published Patent Application No. 2002/0173478 to Gewirtz and in U.S. Published Patent Application No. 2004/0018176 to Reich et al., the entire disclosures of which are incorporated herein by reference.

Expression of a given gene can also be inhibited by an antisense nucleic acid. As used herein, an “antisense nucleic acid” refers to a nucleic acid molecule that binds to target RNA by means of RNA-RNA or RNA-DNA or RNA-peptide nucleic acid interactions, which alters the activity of the target RNA. Antisense nucleic acids suitable for use in the present methods are single-stranded nucleic acids (e.g., RNA, DNA, RNA-DNA chimeras, PNA) that generally comprise a nucleic acid sequence complementary to a contiguous nucleic acid sequence in a gene product. The antisense nucleic acid can comprise a nucleic acid sequence that is 50-100% complementary, 75-100% complementary, or 95-100% complementary to a contiguous nucleic acid sequence in a gene product. Nucleic acid sequences for the gene products are provided herein. Without wishing to be bound by any theory, it is believed that the antisense nucleic acids activate RNase H or another cellular nuclease that digests the gene product/antisense nucleic acid duplex.

Antisense nucleic acids can also contain modifications to the nucleic acid backbone or to the sugar and base moieties (or their equivalent) to enhance target specificity, nuclease resistance, delivery or other properties related to efficacy of the molecule. Such modifications include cholesterol moieties, duplex intercalators, such as acridine, or one or more nuclease-resistant groups.

Antisense nucleic acids can be produced chemically or biologically, or can be expressed from a recombinant plasmid or viral vector, as described above for the isolated gene products. Exemplary methods for producing and testing are within the skill in the art; see, e.g., Stein and Cheng (1993), Science 261:1004 and U.S. Pat. No. 5,849,902 to Woolf et al., the entire disclosures of which are incorporated herein by reference.

Expression of a given gene can also be inhibited by an enzymatic nucleic acid. As used herein, an “enzymatic nucleic acid” refers to a nucleic acid comprising a substrate binding region that has complementarity to a contiguous nucleic acid sequence of a gene product, and which is able to specifically cleave the gene product. The enzymatic nucleic acid substrate binding region can be, for example, 50-100% complementary, 75-100% complementary, or 95-100% complementary to a contiguous nucleic acid sequence in a gene product. The enzymatic nucleic acids can also comprise modifications at the base, sugar, and/or phosphate groups. An exemplary enzymatic nucleic acid for use in the present methods is a ribozyme.

The enzymatic nucleic acids can be produced chemically or biologically, or can be expressed from a recombinant plasmid or viral vector, as described above for the isolated gene products. Exemplary methods for producing and testing dsRNA or siRNA molecules are described in Werner and Uhlenbeck (1995), Nucl. Acids Res. 23:2092-96; Hammann et al. (1999), Antisense and Nucleic Acid Drug Dev. 9:25-31; and U.S. Pat. No. 4,987,071 to Cech et al, the entire disclosures of which are incorporated herein by reference.

Administration of at least one gene product, or at least one compound for inhibiting expression, will inhibit the proliferation of cancer cells in a subject who has a cancer associated with a cancer-associated chromosomal feature. As used herein, to “inhibit the proliferation of a cancer cell” means to kill the cell, or permanently or temporarily arrest or slow the growth of the cell. Inhibition of cancer cell proliferation can be inferred if the number of such cells in the subject remains constant or decreases after administration of the gene products or gene expression-inhibiting compounds. An inhibition of cancer cell proliferation can also be inferred if the absolute number of such cells increases, but the rate of tumor growth decreases.

The number of cancer cells in a subject's body can be determined by direct measurement, or by estimation from the size of primary or metastatic tumor masses. For example, the number of cancer cells in a subject can be measured by immunohistological methods, flow cytometry, or other techniques designed to detect characteristic surface markers of cancer cells.

The gene products or gene expression-inhibiting compounds can be administered to a subject by any means suitable for delivering these compounds to cancer cells of the subject. For example, the gene products or expression inhibiting compounds can be administered by methods suitable to transfect cells of the subject with these compounds, or with nucleic acids comprising sequences encoding these compounds. In one embodiment, the cells are transfected with a plasmid or viral vector comprising sequences encoding at least one gene product or gene expression inhibiting compound.

Transfection methods for eukaryotic cells are well known in the art, and include, e.g., direct injection of the nucleic acid into the nucleus or pronucleus of a cell; electroporation; liposome transfer or transfer mediated by lipophilic materials; receptor-mediated nucleic acid delivery, bioballistic or particle acceleration; calcium phosphate precipitation, and transfection mediated by viral vectors.

For example, cells can be transfected with a liposomal transfer compound, e.g., DOTAP (N-[1-(2,3-dioleoyloxy)propyl]-N,N,N-trimethyl-ammonium methylsulfate, Boehringer—Mannheim) or an equivalent, such as LIPOFECTIN. The amount of nucleic acid used is not critical to the practice of the invention; acceptable results may be achieved with 0.1-100 micrograms of nucleic acid/10⁵ cells. For example, a ratio of about 0.5 micrograms of plasmid vector in 3 micrograms of DOTAP per 10⁵ cells can be used.

A gene product or gene expression inhibiting compound can also be administered to a subject by any suitable enteral or parenteral administration route. Suitable enteral administration routes for the present methods include, e.g., oral, rectal, or intranasal delivery. Suitable parenteral administration routes include, e.g., intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection, intra-retinal injection, or subretinal injection); subcutaneous injection or deposition, including subcutaneous infusion (such as by osmotic pumps); direct application to the tissue of interest, for example by a catheter or other placement device (e.g., a retinal pellet or a suppository or an implant comprising a porous, non-porous, or gelatinous material); and inhalation. Particularly suitable administration routes are injection, infusion and intravenous administration into the patient.

In the present methods, a gene product or gene product expression inhibiting compound can be administered to the subject either as naked RNA, in combination with a delivery reagent, or as a nucleic acid (e.g., a recombinant plasmid or viral vector) comprising sequences that express the gene product or expression inhibiting compound. Suitable delivery reagents include, e.g., the Mirus Transit TKO lipophilic reagent; lipofectin; lipofectamine; cellfectin; polycations (e.g., polylysine), and liposomes.

Recombinant plasmids and viral vectors comprising sequences that express the gene products or gene expression inhibiting compounds, and techniques for delivering such plasmids and vectors to cancer cells, are discussed herein.

In a particular embodiment, liposomes are used to deliver a gene product or gene expression-inhibiting compound (or nucleic acids comprising sequences encoding them) to a subject. Liposomes can also increase the blood half-life of the gene products or nucleic acids. Suitable liposomes for use in the invention can be formed from standard vesicle-forming lipids, which generally include neutral or negatively charged phospholipids and a sterol, such as cholesterol. The selection of lipids is generally guided by consideration of factors, such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are incorporated herein by reference.

The liposomes for use in the present methods can comprise a ligand molecule that targets the liposome to cancer cells. Ligands which bind to receptors prevalent in cancer cells, such as monoclonal antibodies that bind to tumor cell antigens, are preferred.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. In a particularly preferred embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is incorporated herein by reference.

Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers, such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or derivatives thereof. Liposomes modified with PEG or PEG-derivatives are sometimes called “PEGylated liposomes.”

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects, for example solid tumors, will efficiently accumulate these liposomes; see Gabizon, et al. (1988), Proc. Natl. Acad. Sci., U.S.A., 18:6949-53. In addition, the reduced uptake by the RES lowers the toxicity of stealth liposomes by preventing significant accumulation of the liposomes in the liver and spleen. Thus, liposomes that are modified with opsonization-inhibition moieties are particularly suited to deliver the gene products or gene expression inhibition compounds (or nucleic acids comprising sequences encoding them) to tumor cells.

The gene products or gene expression inhibition compounds can be formulated as pharmaceutical compositions, sometimes called “medicaments,” prior to administering them to a subject, according to techniques known in the art. Accordingly, the invention encompasses pharmaceutical compositions for treating MM and/or MGUS. In one embodiment, the pharmaceutical compositions comprise at least one isolated gene product and a pharmaceutically-acceptable carrier. In a particular embodiment, the at least one gene product corresponds to a gene product that has a decreased level of expression in MM and/or MGUS cells relative to suitable control cells.

In other embodiments, the pharmaceutical compositions of the invention comprise at least one expression inhibition compound. In a particular embodiment, the at least one gene expression inhibition compound is specific for a gene whose expression is greater in MM and/or MGUS cells than control cells.

Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is incorporated herein by reference.

The present pharmaceutical formulations comprise at least one gene product or gene expression inhibition compound (or at least one nucleic acid comprising sequences encoding them) (e.g., 0.1 to 90% by weight), or a physiologically acceptable salt thereof, mixed with a pharmaceutically-acceptable carrier. The pharmaceutical formulations of the invention can also comprise at least one gene product or gene expression inhibition compound (or at least one nucleic acid comprising sequences encoding them) which are encapsulated by liposomes and a pharmaceutically-acceptable carrier.

Especially suitable pharmaceutically-acceptable carriers are water, buffered water, normal saline, 0.4% saline, 0.3% glycine, hyaluronic acid and the like.

In a particular embodiment, the pharmaceutical compositions of the invention comprise at least one gene product or gene expression inhibition compound (or at least one nucleic acid comprising sequences encoding them) which is resistant to degradation by nucleases. One skilled in the art can readily synthesize nucleic acids which are nuclease resistant, for example by incorporating one or more ribonucleotides that are modified at the 2′-position into the gene products. Suitable 2′-modified ribonucleotides include those modified at the 2′-position with fluoro, amino, alkyl, alkoxy, and O-allyl.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include, e.g., physiologically biocompatible buffers (e.g., tromethamine hydrochloride), additions of chelants (such as, for example, DTPA or DTPA-bisamide) or calcium chelate complexes (such as, for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid pharmaceutical compositions of the invention, conventional nontoxic solid pharmaceutically-acceptable carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25%-75%, of the at least one gene product or gene expression inhibition compound (or at least one nucleic acid comprising sequences encoding them). A pharmaceutical composition for aerosol (inhalational) administration can comprise 0.01-20% by weight, preferably 1%-10% by weight, of the at least one gene product or gene expression inhibition compound (or at least one nucleic acid comprising sequences encoding them) encapsulated in a liposome as described above, and a propellant. A carrier can also be included as desired; e.g., lecithin for intranasal delivery.

The invention also encompasses methods of identifying an anti-ALL agent, comprising providing a test agent to a cell and measuring the level of at least one gene product in the cell. In one embodiment, the method comprises providing a test agent to a cell and measuring the level of at least one gene product associated with decreased expression levels in MM and/or MGUS cells. An increase in the level of the gene product in the cell, relative to a suitable control cell, is indicative of the test agent being an anti-ALL agent. In a particular embodiment, at least one gene product associated with decreased expression levels in MM and/or MGUS cells is selected from the group described herein and combinations thereof.

In other embodiments the method comprises providing a test agent to a cell and measuring the level of at least one gene product associated with increased expression levels in MM and/or MGUS cells. A decrease in the level of the gene product in the cell, relative to a suitable control cell, is indicative of the test agent being an anti- MM and/or MGUS agent. In a particular embodiment, at least one gene product associated with increased expression levels in MM and/or MGUS cells is selected from the groups described herein and combinations thereof.

Suitable agents include, but are not limited to drugs (e.g., small molecules, peptides), and biological macromolecules (e.g., proteins, nucleic acids). The agent can be produced recombinantly, synthetically, or it may be isolated (i.e., purified) from a natural source. Various methods for providing such agents to a cell (e.g., transfection) are well known in the art, and several of such methods are described hereinabove. Methods for detecting the expression of at least one gene product (e.g., Northern blotting, in situ hybridization, RT-PCR, expression profiling) are also well known in the art.

### DEFINITIONS

The term “array” is used interchangeably with the term “microarray” herein.

The term “cancer,” as used herein, refers to the physiological condition in mammals that is typically characterized by unregulated cell proliferation, and the ability of those cells to invade other tissues.

The term “expression,” as used herein, refers to the conversion of the DNA sequence information into messenger RNA (mRNA) or protein. Expression may be monitored by measuring the levels of full-length mRNA, mRNA fragments, full-length protein, or protein fragments.

The phrase “gene expression signature,” as used herein refers to the unique pattern of gene expression in a cell, and in particular, a cancer cell.

The term “hybridization,” as used herein, refers to the process of binding, annealing, or base-pairing between two single-stranded nucleic acids. The “stringency of hybridization” is determined by the conditions of temperature and ionic strength. Nucleic acid hybrid stability is expressed as the melting temperature or Tm, which is the temperature at which the hybrid is 50% denatured under defined conditions. Equations have been derived to estimate the Tm of a given hybrid; the equations take into account the G+C content of the nucleic acid, the length of the hybridization probe, etc. (e.g., Sambrook et al., 1989). To maximize the rate of annealing of the probe with its target, hybridizations are generally carried out in solutions of high ionic strength (6×SSC or 6×SSPE) at a temperature that is about 2025° C. below the Tm. If the sequences to be hybridized are not identical, then the hybridization temperature is reduced 1-1.5° C. for every 1% of mismatch. In general, the washing conditions should be as stringent as possible (i.e., low ionic strength at a temperature about 12-20° C. below the calculated Tm). As an example, highly stringent conditions typically involve hybridizing at 68° C. in 6×SSC/5× Denhardt's solution/1.0% SDS and washing in 0.2×SSC/0.1% SDS at 65° C. The optimal hybridization conditions generally differ between hybridizations performed in solution and hybridizations using immobilized nucleic acids. One skilled in the art will appreciate which parameters to manipulate to optimize hybridization.

The term “nucleic acid,” as used herein, refers to sequences of linked nucleotides. The nucleotides may be deoxyribonucleotides or ribonucleotides, they may be standard or non-standard nucleotides; they may be modified or derivatized nucleotides; they may be synthetic analogs. The nucleotides may be linked by phosphodiester bonds or non-hydrolyzable bonds. The nucleic acid may comprise a few nucleotides (i.e., oligonucleotide), or it may comprise many nucleotides (i.e., polynucleotide). The nucleic acid may be single-stranded or double-stranded.

The term “prognosis,” as used herein refers to the probable course and outcome of a cancer, and in particular, the likelihood of recovery.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

### REFERENCES

The publication and other material used herein to illuminate the invention or provide additional details respecting the practice of the invention, are incorporated be reference herein, and for convenience are provided in the following bibliography.

Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

1. Bommert K, Bargou R, Stühmer T (2006) Signalling and survival pathways in multiple myeloma. Eur J Cancer 42:1574-1580.

2. Fonseca R, San Miguel J (2007) Prognostic factors and staging in multiple myeloma. Hematol Oncol Clin N Am 21: 1115-40

3. Hideshima T, Mitsiades C, Tono G, Richardson P G and Anderson K C. (2007) Understanding multiple myeloma pathogenesis in the bone marrow to identify new therapeutic targets. Nature Reviews Cancer 7:585-598.

4. Oancea M, Mani A, Hussein M A, Almasan A (2004) Apoptosis of multiple myeloma. Int J Hematol 80:224-231.

5. Drach J, Schuster J, Nowotny H, et al. (1995) Multiple myeloma: high incidence of chromosomal aneuploidy as detected by interphase fluorescence in situ hybridization. Cancer Res 55:3854-3859.

6. Latreille J, Barlogie B, Johnston D, Drewinko B, Alexanian R (1982) Ploidy and proliferative characteristics in monoclonal gammopathies. Blood 59:43-51.

7. Chng W J, Glebov O, Bergsagel P L, Kuehl W M. (2007) Genetic events in the pathogenesis of multiple myeloma. Best Pract Res Clin Haematol. 20:571-96.

8. Calin G A, et al. (2005) A microRNA signature associated with prognosis and progression in chronic lymphocytic leukemia. N Engl J Med 353:1793-1801.

9. Garzon R, et al. (2008) MicroRNA signatures associated with cytogenetics and prognosis in acute myeloid leukemia Blood 111:3183-3189.

10. Bartel D (2004) MicroRNAs: Genomics, biogenesis, mechanism, and function. Cell 116:281-297.

11. Croce C M (2008) Oncogenes and cancer. N Engl J Med 358:502-511.

12. Garzon R, et al. (2007) MicroRNA gene expression during retinoic acid-induced differentiation of human acute promyelocytic leukemia. Oncogene 26:4148-4157.

13. Calin G A, Croce C M (2006) MicroRNA signatures in human cancers. Nat Rev Cancer 6:857-866.

14. Löffler D, et al. (2007) Interleukin-6 dependent survival of multiple myeloma cells involves the Stat3-mediated induction of microRNA-21 through a highly conserved enhancer. Blood 110:1330-1333.

15. Seidl S, Kaufmann H, Drach J (2003) New insights into the pathophysiology of multiple myeloma. Lancet Oncol 4:557-64.

16. Chen C Z, Li L, Lodish H F, Bartel D P (2004) MicroRNAs modulate hematopoietic lineage differentiation. Science 303:83-86.

17. Liu C G, Calin G A, Volinia S, Croce C M (2008) MicroRNA expression profiling using microarrays. Nat Protoc 3:563-578.

18. He L, Thomson, et al. (2005). A microRNA polycistron as a potential human oncogene. Nature 435:828-833.

19. Ventura A, et al. (2008) Targeted deletion reveals essential and overlapping functions of the miR-17 through 92 families of miRNA clusters. Cell 132:875-886.

20. Petrocca F, et al. (2008) E2F1-regulated microRNAs impair TGFbeta-dependent cell-cycle arrest and apoptosis in gastric cancer. Cancer Cell 13:272-86.

21. Lewis B, Shih I, Jones-Rhoades M, Bartel D, Burge C (2003) Prediction of mammalian microRNA targets. Cell 115:787-798.

22. Krek A, et al. (2005) Combinatorial microRNA target prediction. Nat Genet 37:495-500.

23. Schiltz R L, Nakatani Y (2000) The PCAF acetylase complex as a potential tumor suppressor. Biochim Biophys Acta 1470:M37-M53.

24. Linares L K, et al. (2007) Intrinsic ubiquitination activity of PCAF controls the stability of the oncoprotein Hdm2. Nat Cell Biol 9:331-338.

25. Imamura J, Miyoshi I, Koeffler H P (1994) p53 in hematologic malignancies. Blood 84:2412-21.

26. Stühmer T, Bargou R C Selective pharmacologic activation of the p53-dependent pathway as a therapeutic strategy for hematologic malignancies. Cell Cycle 5:39-42.

27. Greenhalgh C J, Hilton D J (2001) Negative regulation of cytokine signaling. J Leukoc Biol 70:348-356.

28. Galm O, Yoshikawa H, Esteller M, Osieka R, Herman J G (2002) SOCS-1, a negative regulator of cytokine signaling, is frequently silenced by methylation in multiple myeloma. Blood 101:2784-8.

29. Schwab G, Siegall C B, Aarden L A, Neckers L M, Nordan R P (1991) Characterization of an interleukin-6-mediated autocrine growth loop in the human multiple myeloma cell line, U266. Blood 77:587-93.

30. Volinia S, et al. (2006) A microRNA expression signature of human solid tumors defines cancer gene targets. Proc Natl Acad Sci USA 103:2257-61.

31. Garzon R, et al. (2008) Distinctive microRNA signature of acute myeloid leukemia bearing cytoplasmic mutated nucleophosmin. Proc Natl Acad Sci USA 105:3945-5.

32. Zhan F, et al. (2002) Global gene expression profiling of multiple myeloma, monoclonal gammopathy of undetermined significance, and normal bone marrow plasma cells. Blood, 99:1745-1757.

33. Chan J A, Krichevsky A M, Kosik K S. (2005) MicroRNA-21 is an antiapoptotic factor in human glioblastoma cells. Cancer Res 65:6029-33.

34. Meng F, et al. (2007) MicroRNA-21 regulates expression of the PTEN tumor suppressor gene in human hepatocellular cancer. Gastroenterology 133:647-58.

35. Novotny G W, et al. (2007). Translational repression of E2F1 mRNA in carcinoma in situ and normal testis correlates with expression of the miR-17-92 cluster. Cell Death Difer 14: 879-882.

36. O'Donnell K A, Wentzel E A, Zeller K I, Dang C V, and Mendell, J T (2005). c-Myc-regulated microRNAs modulate E2F1 expression. Nature 435: 839-843.

37. Liu C G, et al. (2004) An oligonucleotide microchip for genomic-wide microRNA profiling in human and mouse tissues. Proc Natl Acad Sci USA 101:9740-9744.

1. A method of diagnosing whether a subject has, or is at risk for developing, multiple myeloma (MM) as compared to monoclonal gammopathy of undetermined significance (MGUS), comprising:
measuring the level of at least one miR gene product in a test sample from the subject, wherein the at least one miR gene product is one or more of miR-17-19 cluster and miR-32, and
wherein an alteration in the level of the miR gene product in the test sample, relative to the level of a corresponding miR gene product in a control sample, is indicative of the subject either having, or being at risk for developing, MM.

2. The method of claim 1, wherein the at least one miR gene product is one or more of: miR-17˜92 cluster, miR-19a, miR-19b and miR-32.

3. The method of claim 1, wherein the at least one miR gene product is one or more of: miR-19a, miR-19b, and miR-32.

4. The method of claim 1, wherein the at least one miR gene product is one or more of miR-19a and miR-19b.

5. (canceled)

6. (canceled)

7. (canceled)

8. (canceled)

9. The method of claim 1, wherein at least one miR gene product is associated with p53 protein regulation.

10. A method of diagnosing whether a subject has, or is at risk for developing, multiple myeloma (MM), comprising:
(1) reverse transcribing RNA from a test sample obtained from the subject to provide a set of target oligodeoxynucleotides;
(2) hybridizing the target oligodeoxynucleotides to a microarray comprising miRNA-specific probe oligonucleotides to provide a hybridization profile for the test sample; wherein the at least one miRA is one or more of miR-17-19 cluster and miR-32; and
(3) comparing the test sample hybridization profile to a hybridization profile generated from a control sample,
wherein an alteration in the signal of at least one miRNA is indicative of the subject either having, or being at risk for developing, the MM disease.

11. (canceled)

12. (canceled)

13. (canceled)

14. The method of claim 10, wherein the at least one miR is one or more of: miR-17˜92 cluster, miR-19a, miR-19b and miR-32.

15. The method of claim 10, 11, 12 or 13, wherein the at least one miR is one or more of: miR-19a, miR-19b and miR-32.

16. The method of claim 10, wherein the at least one miR is one or more of miR-19a and miR-19b.

17. The method of claim 10, wherein the at least one miR is indicative of the subject having MM, as distinguished from MGUS.

18. A pharmaceutical composition for treating a multiple myeloma (MM) related disease, comprising at least one isolated miR gene product and a pharmaceutically-acceptable carrier, wherein the least one miR gene product is one or more of: miR-17˜92 cluster, miR-19a, miR-19b and miR-32.

19. (canceled)

20. The pharmaceutical composition of claim 18, wherein the at least one miR gene product is one or more of: miR-19a, miR-19b, and miR-32.

21. The pharmaceutical composition of claim 18, wherein the at least one miR gene product is one or more of miR-19a and miR-19b.

22. The pharmaceutical composition of claim 18, wherein the miR gene product comprises at least one or more of: miR expression inhibitors and anti-sense oligos (ASOs).

23. The pharmaceutical composition of claim 18, wherein the at least one miR expression inhibitor compound is specific for a miR gene product that is up-regulated in MM cells relative to suitable control cells.

24. A method of identifying an anti-multiple myeloma (MM) related disease agent, comprising:
providing a test agent to a cell, and
measuring the level of at least one miR gene product associated with altered expression levels in MM cells, wherein the at least one miR gene product is one or more of miR-17-19 cluster and miR-32, and
wherein the alteration in the level of the miR gene product in the cell, relative to a suitable control cell, is indicative of the test agent being an anti-cancer agent.

25. The method of claim 24, wherein the at least one miR gene product is one or more of: miR-17˜92 cluster, miR-19a, miR-19b and miR-32.

26. The method of claim 24, wherein the at least one miR gene product is one or more of: miR-19a, miR-19b and miR-32.

27. The method of claim 24, wherein the at least one miR gene product is one or more of miR-19a and miR-19b.

28. A marker for assessing one or more metabolic pathways that contribute to at least one of initiation, progression, severity, pathology, aggressiveness, grade, activity, disability, mortality, morbidity, disease sub-classification or other underlying pathogenic or pathological feature of a multiple myeloma (MM) related disease,
wherein the marker comprises one or more gene products coding for least one isolated miR gene product is miR-17˜92 cluster, miR-19a, miR-19b and miR-32.

29. The marker of claim 28, wherein the at least one miR gene product is one or more of: miR-19a, miR-19b and miR-32.

30. The marker of claim 28, wherein the at least one miR gene product is one or more of miR-19a and miR-19b.

31. (canceled)

32. A reagent for testing for a multiple myeloma (MM) related disease, wherein the reagent comprises an antibody that recognizes a protein encoded by at least one marker of claim 28.

33. A method of assessing the effectiveness of a therapy to prevent, diagnose and/or treat multiple myeloma (MM)comprising:
(1) subjecting a subject to a therapy whose effectiveness is being assessed, and
(2) determining the level of effectiveness of the treatment being tested in treating or preventing multiple myeloma (MM) by evaluating at least one marker of claim 28.

34. The method of claim 33, wherein the candidate therapeutic agent comprises one or more of: pharmaceutical compositions, nutraceutical compositions, and homeopathic compositions.

35. The method of claim 33, wherein the therapy being assessed is for use in a human subject.

36. A kit for screening for a candidate compound for a therapeutic agent to treat a multiple myeloma (MM) related disease, wherein the kit comprises: one or more reagents of at least one marker of claim 28, and a cell expressing at least one marker.

37. The kit of claim 36, wherein the presence of the marker is detected using a reagent comprising an antibody or an antibody fragment which specifically binds with at least one marker.

38. A screening test for multiple myeloma (MM) comprising: contacting one or more of the markers of claim 28, with a substrate for such marker and with a test agent; and, determining whether the test agent modulates the activity of the marker.

39. The screening test of claim 38, wherein all method steps are performed in vitro.

40. (canceled)

41. Use of an agent that interferes with at a multiple myeloma (MM) response signaling pathway, for the manufacture of a medicament for treating, preventing, reversing or limiting the severity of a multiple myeloma (MM) complication in an individual,
wherein the agent comprises at least one gene product selected from a group consisting at least one miR gene product of miR-17˜92 cluster, miR-19a, miR-19b, and miR-32 and combinations thereof.

42. (canceled)

43. (canceled)

44. (canceled)

45. (canceled)

46. An miRNA signature associated with a MM multi step transformation process from normal to via MGUS to clinically overt MM, comprising: at least one or more of miR-17˜92 cluster, miR-19a, miR-19b and miR-32.

47. The miRNA signature of claim 46, wherein the at least one miR gene product is one or more of: miR-19a, miR-19b, and miR-32.

48. The miRNA signature of claim 46, wherein the at least one miR gene product is one or more of miR-19a and miR-19b.

49. (canceled)

50. (canceled)

51. A method for downregulating SOCS-1 and/or activating IL-6 in later stages in MM pathogenesis in a cell, comprising up-regulating miR-19s in the cell.
